(19) United States
(12) Patent Application Publication
(10) Pub. No.: US 2023/0323358 A1
(43) Pub. Date: Oct. 12, 2023

(54) COMPOSITION AND USE OF siRNAs AGAINST VEGFR2 AND TGF-BETA1 IN COMBINATION THERAPY FOR CANCER

(71) Applicant: Sirnaomics, Inc., Germantown, MD (US)

(72) Inventors: John XU, Suzhou (CN); Zhiyuan WANG, Suzhou (CN); Deling WANG, Suzhou (CN); Patrick Y. LU, Germantown, MD (US); Wanying JIA, Suzhou (CN); Jin ZHANG, Suzhou (CN); Xudong ZHU, Suzhou (CN); Jingming ZHANG, Suzhou (CN)

(21) Appl. No.: 18/179,573

(22) Filed: Mar. 7, 2023

Related U.S. Application Data
(63) Continuation of application No. PCT/US2022/037519, filed on Jul. 18, 2022.
(60) Provisional application No. 63/222,418, filed on Jul. 16, 2021.

(30) Foreign Application Priority Data
Jul. 16, 2021 (CN) 202110806912.8
Jul. 15, 2022 (CN) 2022108362378

(51) Int. Cl. C12N 15/113; A61K 9/51; A61P 35/00

(52) U.S. Cl. CPC C12N 15/1136; A61K 9/5169; A61P 35/00; C12N 15/1138; C12N 2310/14; C12N 2310/3513

(57) ABSTRACT

The techniques of the present disclosure provide a method of inhibiting tumor growth in a tissue of a mammal. The method includes administering to the mammal a therapeutically effective amount of a composition comprising an siRNA molecule that binds to an mRNA that codes for TGFβ1 protein, an siRNA molecule that binds to an mRNA that codes for VEGFR2 protein, and a pharmaceutically acceptable carrier comprising a pharmaceutically acceptable polypeptide polymer. The techniques of the present disclosure also provide for additional methods for using this composition.

### RELATED APPLICATIONS

This application is a continuation of International Application No. PCT/US22/37519, filed Jul. 18, 2022, claiming priority to U.S. Provisional Application No. 63/222,418, filed Jul. 16, 2021 and Chinese provisional application 202110806912.8, filed Jul. 16, 2021, each of which is incorporated herein by reference in its entirety.

### SEQUENCE LISTING

The instant application contains a Sequence Listing which has been submitted electronically in XML format and is hereby incorporated by reference in its entirety. Said XML (ST.26) copy, created on May 22, 2023, is named 46900045C_sequence_list_ST.26 and is 322 kilobytes in size.

### FIELD

Compositions containing combinations of siRNA molecules are provided, together with nanoparticle carriers and drug formulations containing the compositions. Methods are also provided for treatment of cancers including pancreatic, breast, and prostate cancer using multiple siRNAs administered in formulations with polypeptides.

### BACKGROUND

### VEGF/VEGFR2 Signaling Pathway Promotes Neovasculature Formation in Tumor Tissues

Angiogenesis, or neovasculature formation, is an integral part of homeostasis regulation networks as blood vessels are pathways to cells of nutrient delivery and waste disposal. Angiogenesis occurs across the process of organism development, regeneration from injuries, as well as in many tumorigeneses. The concept of the involvement of angiogenesis in tumorigenesis was proposed more than 7 decades ago by Ide and others (Ide et al., 1939); Algire et al., 1945) when it was observed that robust growth of new blood vessels in tumor tissues could be stimulated by “blood-vessel growth-stimulating factors” which rendered a growth advantage to tumor cells. Interest in the field was rekindled by Folkman’s proposal in 1971 that angiogenesis inhibitors could be applied to treat cancers and other related disorders. (Folkman, 1971)

Angiogenesis takes place in normal physiological processes and is precisely controlled by a number of regulators, but mainly by vascular endothelial-derived growth factors (VEGFs). This is a family of proteins that includes VEGF-A, VEGF-B, VEGF-C, VEGF-D, VEGF-E and VEGF -F (VEG-F165 as the most dominant isoform in tissue). The main target cells for VEGF are endothelial cells, although other cells also are targets.. VEGF functions through binding with its receptors (VEGFR1, VEGFR2, and VEGFR3) on cell membranes. VEGF receptors are receptor tyrosine kinases (RTK) consisting of an extracellular seven immunoglobulin-homology domain, a transmembrane domain, and an intracellular regulatory tyrosine kinase domain, and are expressed mainly on endothelial cell membranes. After binding to a receptor, VEGF triggers a series of signal transducing effects, stimulating endothelial cell proliferation, migration and new vessel formation.

Even though VEGFs and their receptors are essential to maintaining homeostasis in many tissues, their function has been studied mainly in pathological processes such as tumorigenesis and hypertrophic scar formation despite the fact that FEGF/VEGFR are physiologically essential in maintaining homeostasis in many normal tissues. VEGF production increases only in response to tissue hypoxia under normal situations. In most cancers, however, VEGF is overexpressed (Kerbel, 2008); and is associated with structurally aberrant neo-angiogenesis within tumors and their surrounding tissues as angiogenesis is required to meet enhanced nutritional demand for uncontrolled tumor proliferation. (Ferrara, 2010; Jain, 2003; Nagy et al., 2009). Insights derived from studies of VEGF functionality led to therapeutic strategies targeting the VEGF/VEGFR signaling pathway. For example, Avastin, which inhibits VEGF-A, is only one of the successful drugs. (Ferrara and Adamis, 2016; Jayson et al., 2016). This humanized monoclonal antibody was widely applied in many cancer therapies, including colon cancer, non-squamous non-small cell lung carcinoma (NSCLC), renal cell carcinoma (RCC), glioblastoma multiforme, ovarian cancer, and cervical cancer. Treatments using combinations of drugs, however, have become common, and reflect the standard of care in current cancer therapies. Simultaneously targeting multiple peptides or proteins is considered to be more effective (Gerber and Ferrara, 2005). Preclinical studies have consistently shown additive or synergistic benefits from combinations of VEGF inhibitors with cytotoxic agents.

### TGFβ Is a Key Player During Tumorigenesis

TGFβs are a group are pleiotropic cytokines participating in basic physiological processes such as proliferation, differentiation, metabolism, and apoptosis. Homeostasis of multicellular organisms, including mammals, is maintained and regulated by complicated networks of hormones and cytokines TGFβs are present only in mammals, among which TGFβ1 is the most abundantly and ubiquitously expressed. Although reportedly able to exert distal effects, TGFβ1 functions basically as an effector at the locales where it is stored in the extracellular matrix after being secreted mainly as a latent complex. (Crane & Cao, 2014; Annes et al, 2003). Evidence has indicated that TGFβ can respond to injuries, causing inflammation in local tissue and acts swiftly to restore local extracellular matrix homeostasis. (Annes, supra). Therefore, the temporal and spatial activation of this growth factor plays a critical role in its context-dependent physiological effects in vivo.

There are three transforming growth factor β receptors (TGFβR): TGFβR1, TGFβR2, and TGFβR3. (Bierie and Moses, 2006). TGFβ ligands function through binding to the heterotetrameric complex of TGFβR receptors. Both TGFβR1s and two TGFβR2 exhibit serine/threonine kinase activity and are involved in transducing signals through downstream component molecules, or Smads (Wrana, et al, 1994.; ten Dijke, et al. 1994).

As a pleiotropic regulator of homeostasis the TGFβ/TGFβR2 receptor signal transduction is usually transmitted through the canonical signal transduction pathway to regulate downstream molecules, Smads. TGF-β/TβRII receptor signal transduction also activates members of the mitogen-activated protein (MAP) kinase signaling pathway; these include: JNK, p38, ERKs, and the PI3 K/AKT (Ikushima and Miyazono, 2010), and activation is through the noncanonical signal transduction pathway.

TGFβ has been implicated as an important player in tumorigenesis and tumor progression, and the term “transforming” in its title refers to its ability to transform normal fibroblast from a phenotype of anchorage-dependent cell growth to a phenotype of anchorage independent cell colonies in soft agar, a hallmark of tumorigenesis. (Keski-Oja et al., 1987). More recent studies have shown that TGFβ can act either as a potent inhibitor of cell proliferation in early premalignant growth (Roberts & Wakefield, 2003; Adam et al; 1994), or as a promotor of tumor cell migration and proliferation in late-stage progression and metastatic cancer (Lu, et al., 1999). Loss of TGFβ growth inhibition and increased expression of TGFβ have been associated with malignant conversion and progression in many tissues/organ cancers, including gliomas and melanomas, and breast, gastric, endometrial, ovarian, cervical cancers, glioma and melanoma. Studies show that, upon accumulation of genetic and epigenetic alterations in tumor cells, a progressive increase in locally secreted TGFβ levels promote tumor growth by evading immunosurveillance, stimulating connective tissue formation and angiogenesis, and stimulating epithelial-mesenchymal transformation (EMT), which promote invasion and metastasis. An important aberration in the TGFB signal transduction mechanism has also been revealed: stimulation of the noncanonical signal transduction pathway, leading to induction of VEGF through the MEK-Erk and p38 pathways in colon cancer progression and drug resistance (Papageorgis et al., 2011).

### Both TGFβ and VEGF Are Essential Players Eliciting Immunotolerance in TME Cooperatively

Elevated levels of TGFβ in serum are often observed in the later stages of cancer in patients, proposed as a compensatory reaction to the lost TGFβ suppressive effects. (Gold, 1999). However, increased TGFβ could induce proliferation of regulatory T cells (Tregs). Tregs in the tumor microenvironment (TME) induce T cell inertial focusing and exhaustion and facilitates immune tolerance (Fontenot et al., 2003; Yamagiwa et al., 2001). Nakamura et al. reported that overexpression of TGFβ in CT26 colorectal carcinoma cells enhanced tumor growth by suppressing antitumor T lymphocyte response in immune competent Balb/c mice. (Nakamura et al. 2014) At this stage tumor cells may escape from TGFβ-mediated antiproliferative control, either by erratic signal activations via the noncanonical signal transduction pathway or by gaining somatic mutations in components of the TGF-β pathway (Seoane, 2006). For example, TGF-β could increase secretion of MCP-1 (monocyte chemoattractant protein-1, also known as CCL-2) and actively recruit protumorigenic monocytes into TME. (Diaz-Valdes, N., et al., 2011) Reports indicated that anti-PD-1 resistance may be related to increased levels of CCL-2, CCL-7, CCL-8, and CCL-13. Overexpression of these chemotactic genes are regulated by TGFβ activation implicating TGFβ as a key enforcer of immune tolerance and an obstacle that must overcome to achieve optimal efficacy of immune-checkpoint therapy. (Hugo, W. et al., 2016). TGFβ in one aspect help to model inhibitory TME, and in another also mediates the expression, secretion and activation of integrins and VEGF as well as MMPs which stimulate the migration of endothelial cells, thus promoting tumor neo-angiogenesis and metastatic dissemination. (Padua & Massague, 2009; Hagedorn, et al., 2001; Bachmeier, 2001. Pertovaara, et al., 1994; Kang, Y. et al., 2003; de Jong, J. S., et al, 1998; Hasegawa et al., 2001; Schadendorf et al., 1993; Tai and Wang, 2018). Meanwhile, inhibition of TGFβ signaling with TGFβ-neutralizing antibodies can suppress angiogenesis in human breast and prostate cancer, further validating the role of TGFβ plays as a pro-angiogenic factor during tumor. (Tuxhorn, et al., 2002).

### Dual-Targeted Inhibition of VEGF and TGFβ With Specific siRNAs Can Deter Tumor Growth

Studies have shown that homeostasis is regulated and controlled by ever more complicated networks of signal pathways, which interact to compensate for each other’s functionality. However, these seamless intercalated collaborations in cells pose difficult obstacles for single drug therapy. For example, as a pleiotropic cytokine TGFβ can run through signal transduction in canonic arm dependent upon Smads, or in noncanonical signal transducing pathway by MAPK molecules.

Moreover, increasingly, data have shown that treatments intended to prevent or attenuate angiogenesis may result in emergence of more aggressive and invasive tumors (Bergers et al., 2008). Recent reports provide several mechanisms underlying the development of tumor resistance against anti-angiogenic therapy. For example, in non-small cell lung cancer (NSCLC), therapeutic approaches targeting VEGF should be paired with VEGFR-targeted therapy or vice versa, as both pathways are active in NSCLC and can compensate for each other. Clinical trials have been conducted applying dual VEGF/VEGFR inhibition in NSCLC patients. (Koh et al. 2010)

RNAi (RNA interference) is a physiological regulation mechanism of mRNA expression. It is a sequence-specific, post-transcriptional gene silencing (PTGS), mechanism that reduces the expression of target mRNA molecules. siRNA (small interfering RNA) is a short fragment of a double stranded RNA molecule of 13-25 base pairs (bp) in length. The antisense strand of an siRNA duplex can pair to a specific region on an mRNA molecule (the target mRNA) and prevent its translation. Inside the cell, within the cytoplasm the antisense strand will become wedged into a protein particle called an RNA interference silencing complex, or RISC, and will then anneal with the target mRNA. Thereafter the enzymatic component of RISC will cut the mRNA molecule and begin the mRNA degradation process. Introduction of synthetic siRNA exogenously has proven to be an efficient way to control specific genes in laboratory and clinical experiments.

Simultaneous dual targeting can be performed with drugs across different drug categories ranging from small molecules to monoclonal antibodies and nucleic acids (including antisense oligonucleotides (ASOs) and siRNAs). Synthetic siRNAs share similar chemical properties, which make them uniquely advantageous for ease of administration; siRNAs targeting different genes can be administered in the same formulation.

Strategies to pair anti-TGFB/TGFBR2 with other IO-targeting agents are gaining traction. Bintrafusp alfa is an anti-PD-L1/TGFBR2 fusion construct designed by simultaneously blocking both pathways to reduce immune tolerance in TME (Hanne Lind et al., 2020). Bintrafusp alfa prevents tumor cells from undergoing TFGB-induced EMT and makes them more susceptible to other therapies (David, 2017), and recruits NK and T cells to TME and enhances their cytolytic ability against tumor cells (Batlle and Massague, 2019). Finally, it has been shown to mediate enhanced lysis of human tumor cells via an antibody-dependent cell-mediated cytotoxicity (ADCC), (Grenga, 2018).

### BRIEF DESCRIPTION OF THE DRAWINGS

FIGS. 1A-B shows preliminary screening results of candidate 21-mer (FIG. 1A) and 25-mer (FIG. 1B) VEGFR2 siRNA sequences in the MDA-MB-231 cell line;

FIGS. 2A-B shows initial screening results of candidate 21-mer (FIG. 2A) and 25-mer (FIG. 2B) VEGFR2 siRNA sequences in U87MG cell line;

FIG. 3. The preliminary screening results of candidate TGF-β1 siRNA sequences in DLD-1 cell line;

FIG. 4. Preliminary screening results of candidate TGF-β1 siRNA sequences in RKO cell lines;

FIG. 5. The preliminary screening results of candidate TGF-β1 siRNA sequences in U87MG cell line;

FIG. 6. Preliminary screening results of candidate TGF-β1 siRNA sequences in PANC-1 cell line;

FIGS. 7A-B. Comparison of EC50 curves of candidate 21-mer (FIG. 7A), and 21- and 25-mer (FIG. 7B) sequences before and after VEGFR2 siRNA modification in MDA-MB-231 cell line;

FIGS. 8A-B. Comparison of EC50 curves of candidate 21-mer (FIG. 8A), and 21- and 25-mer (FIG. 8B) sequences before and after VEGFR2 siRNA modification in U87MG cell line;

FIGS. 9A-B. Comparison of EC50 curves of candidate 21-mer (FIG. 9A), and 21- and 25-mer (FIG. 9B) sequences before and after VEGFR2 siRNA modification in PANC-1 cell line;

FIGS. 10A-D. Comparison of EC50 curves of candidate sequences before and after TGF-β1 siRNA modification in different cell lines (U87MG (FIG. 10A), PANC-1 (FIG. 10B), RKO (FIG. 10C), BxPC3 (FIG. 10D));

FIGS. 11A-D. Comparison of EC50 curves of candidate sequences before and after TGF-β1 siRNA modification in different cell lines (SK-Hep-1 (FIG. 11A), HUCCT (FIG. 11B), A549 (FIG. 11C), and DLD-1 (FIG. 11D));

FIGS. 12A-B. Comparison of the mass ratio of siRNA molecules of VEGFR2 (FIG. 12A) and TGF-β1 (FIG. 12B) in the composition;

FIGS. 13A-C. In vivo pharmacodynamics of STP355 in mouse pancreatic cancer (PANC-1) xenograft model, showing tumor volume (FIG. 13A), tumor weight (FIG. 13B) and body weight (FIG. 13C) over time;

FIGS. 14A-B. In vivo pharmacodynamics of STP355 in a breast cancer (MDA-MB-231) xenograft tumor mouse model, showing tumor volume (FIG. 14A) and tumor weight (FIG. 14B);

FIGS. 15A-C. In vivo pharmacodynamics of STP355 on humanized PDL1 locus colorectal cancer tumors (MC38-hPDL1) in an immunocompetent mouse model, showing anti-tumor efficiency (FIG. 15A), tumor volumes at IV doses (FIG. 15B) and tumor volume (FIG. 15C);

FIGS. 16A-D. In vivo pharmacodynamics of STP355 on melanoma (B16) in an immunocompetent mouse model, showing anti-tumor efficiency (FIG. 16A), body weight over time (FIG. 16B), tumor weight (FIG. 16C), and tumor size attenuation with STP355 administration (FIG. 16D);

FIGS. 17A-C. Pharmacodynamic comparison test of combination drug and single drug in mouse breast cancer (MDA-MB-231) xenograft model, showing anti-tumor efficiency of three treatment groups (FIG. 17A), anti-tumor efficiency of eight treatment groups (FIG. 17B); tumor volumes for three treatment groups (FIG. 17C);

FIG. 18. In vivo pharmacodynamic comparison of combination drugs and modified drugs in mouse pancreatic cancer (PANC-1) xenograft model;

FIG. 19. Comparison of stability of modified and unmodified drugs in C57BL/6J mice;

FIG. 20 shows histology studies, including staining of TGFβ1. The results showed that TGFβ1 target expression was significantly reduced in both drug administration groups compared to the model control group.

FIG. 21 shows CD31 or TUNEL staining to assess whether inhibition of neo-angiogenesis occurred leading to apoptosis. The expression of CD31, a target associated with angiogenesis, also was significantly reduced in both drug administration groups compared with model control groups. And an increase in the number of apoptotic cells was also observed in both treatment groups.

FIG. 22 shows staining demonstrating cell apoptosis, as detected by TUNEL.

### DETAILED DESCRIPTION

Compositions are provided that contain: a first siRNA molecule that binds to an mRNA that codes for TGFβ1 protein in a mammalian cell; a second siRNA molecule that binds to an mRNA that codes for VEGF protein in a mammalian cell; and a pharmaceutically acceptable carrier comprising a pharmaceutically acceptable polypeptide. Methods of using the compositions for treating also are provided. Advantageously, the carrier is a histidine-lysine copolymer. The composition targets and reduces the expression of at least two different mRNA molecules involved in the VEGF/VEGFR and/or TGFβ pathways.

In one embodiment, a method is provided for dual down-regulating pro-immunotolerance factors and pro-angiogenesis factors in the cells of a mammal, by administering to the mammal a therapeutically effective amount of the composition as described above. In another embodiment, a method is provided for inducing apoptosis in a tumor tissue of a mammal, by administering in the tumor tissue a therapeutically effective amount of the composition. In still another embodiment, a method is provided for reducing the size of a tumor in the tissue of a mammal, comprising administering in the tumor tissue a therapeutically effective amount of the composition. In still another embodiment, a method is provided for reducing tumor size in the tissue of a mammal, comprising co-administering in the tumor tissue a therapeutically effective amount of the composition and a therapeutic monoclonal antibody.

The sequences described below for the siRNA molecules in the composition are the sense strands of double stranded RNA molecules. The double stranded RNA molecules are blunt ended, or may have a one or two base overhang. Advantageously, one or both strands may have one or two deoxyribonucleotide residues at the 3′ end. The skilled artisan will appreciate that the siRNA molecules contain the sense strand (as shown) as part of a duplex with its complementary sequence. Reference herein to the siRNA molecule of, for example, SEQ ID NO. X will be understood to refer to the duplex formed by the sense strand (SEQ ID NO. X) and the corresponding antisense strand.

As used herein, “silencing” a gene means reducing the concentration of the mRNA transcript of that gene such that the concentration of the protein product of that gene is reduced by at least 10%, at least 20%, at least 30%, at least 40%, at least 50%, at least 70%, at least 80% or at least 90% or more. Advantageously, silencing a gene reduces the concentration of the target mRNA to the extent that a desired clinical effect is achieved, for example, shrinkage or elimination of a tumor.

The siRNA molecules may produce additive or synergistic effects in the cells, depending on the compositions and structures of the particular molecules. In a preferred embodiment, they produce a synergistic effect.

As used herein, an “siRNA molecule” is a duplex oligonucleotide, that is a short, doublestranded polynucleotide, that interferes with the expression of a gene in a cell following administration and introduction into the target cells. For example, the siRNA may target and bind to an at least partially complementary nucleotide sequence in a single stranded (ss) target RNA molecule, such as an mRNA or a micro RNA (miRNA). The target mRNA or miRNA is then degraded by the cell.

siRNA molecules may be prepared using techniques known to those skilled in the art.. Examples of such techniques are described in U.S. Pat. Nos. 5, 898,031, 6,107,094, 6,506,559, 7,056,704 and in European Pat. Nos. 1214945 and 1230375, which are incorporated herein by reference in their entireties. By convention in the field, when an siRNA molecule is identified by a particular nucleotide sequence, the sequence refers to the sense strand of the duplex (double stranded) molecule.

The siRNA molecule may be made of naturally occurring ribonucleotides, i.e., those found in living cells, or one or more of its nucleotides may be chemically modified by techniques known in the art, as further described below. In addition to being modified at the level of one or more of its individual nucleotides, the backbone of the oligonucleotide may be modified, for example by replacing one of more phosphodiester molecules with phosphorothioate linkages. Additional modifications include the use of small molecules (e.g. sugar molecules, such as N-acetyl galactosamine), amino acid molecules, peptides, cholesterol, and other large molecules for conjugation onto the siRNA molecule.

In one embodiment, the molecule is an oligonucleotide with a length of about 19 to about 35 base pairs. In one aspect of this embodiment, the molecule is an oligonucleotide with a length of about 19 to about 27 base pairs. In another aspect, the molecule is an oligonucleotide with a length of about 21 to about 25 base pairs. The molecule may have blunt ends at both ends, or sticky ends (an overlapping strand) at each of the ends, or a blunt end at one end of the duplex and a sticky end at the other end of the duplex. Advantageously, one or both strands may have one or two deoxyribonucleotide residues, for example, dT residues, at the 3′ end.

In the compositions as described herein, the relative amounts of the two different molecules and the copolymer may vary. In some embodiments, the ratio of the two different siRNA molecules is about 1:1 by mass. In other embodiments, the ratio of the two different siRNA molecules may be about 1:1 by mass and the ratio of these molecules to the copolymer may be about 1:2.5 by mass. According to the selected ratios, the composition may form nanoparticles with an range in size of about 40-400 nm in diameter.

In one embodiment, the siRNA molecules are selected from those identified in Tables 1 and 2. An example is the pair designated as hmTF-21-hm3# and hmVR2-21-h1#, which have the following sequences:

TF1hm3#:

Sense chain 5'- AACUAUUGCUUCAGCUCCAdTdT- 3' (
SEQ ID No. 23)

Antisense 5' - UGGAGCUGAAGCAAUAGUUdTdT-3' (SE
Q ID No. 33).VEGFR2-21-h1#:

Sense chain, 5'- GCCUAGUGUUUCUCUUGAUdTdT-3' (
SEQ ID No. 60)

Antisense, 5' - AUCAAGAGAAACACUAGGCdTdT -3' (
SEQ ID No. 103).

In each of these siRNA molecules, one or more of the nucleotides in either the sense or the antisense strand can be a modified nucleotide. Modified nucleotides can improve stability and decrease immune stimulation by the siRNAs. The modified nucleotide may be, for example, a 2′-O-methyl, 2′-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino or 2′-O--(N-methylcarbamate) ribonucleotide. Other suitable modifications known in the art.

In addition, one or more of the phosphodiester linkages between the ribonucleotides may be modified to improve resistance to nuclease digestion. Suitable modifications include the use of phosphorothioate and/or phosphorodithioate modified linkages.

### Formation of Nanoparticles Containing siRNAs Targeting VEGFR2 and TGFβ1

The siRNA molecules containing the described above advantageously are formulated into nanoparticles for administration to a subject. Various methods of nanoparticle formation are well known in the art. See, for example, Babu et al., IEEE Trans Nanobioscience, 15: 849-863 (2016).

Advantageously, the nanoparticles are formed using one or more histidine/lysine (HKP) copolymers. Suitable HKP copolymers are described in WO/2001/047496, WO/2003/090719, and WO/2006/060182, the contents of each of which are incorporated herein in their entireties. Examples of suitable HKP polymers include H3K4b (which contains the unit [KH₃]₄K) and H3K4b(+H) (which contains the unit KH₃KH₄[KH₃]₂K). Both H3K4b and H3K4b(+H) have a backbone of three lysine residues where the lysine side chain ε-amino groups and the N-terminus are coupled to the C-terminus of the [KH₃]₄K) or H3K4b(+H) units. The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting VEGFR2 and an siRNA targeting TGFβ1 are mixed with one or more HKP polymers at a fixed flow rate. The flow rate can be varied to vary the size of the nanoparticles produced. HKP copolymers advantageously form a nanoparticle containing an siRNA molecule, typically ~ <100-400 nm in diameter.

Thus, for example, an siRNA targeting VEGFR2 and an siRNA targeting TGFβ1 were mixed at 0.5 mg/ml with HKP(+H) using a PNI microfluidic mixer system (Precision Nanosystems, Inc., Vancouver, CA). Total Flow Rate (TFR) was varied and the effect of this flow rate on particle size was evaluated by measuring resulting particle size using a Malvern Nanosizer system (Malvern Panalytical Inc., Westborough, MA). The polydispersity index (PDI) is an indication of the amount of variation of the nanoparticles around the average size.

In one embodiment, the siRNA molecules are selected from the ones identified in Tables 1 and 2. An illustrative example is the pair designated as hmTF-21-hm3# and hmVR2-21-h2#, which have the following sequences:

TF1hm3#:

Sense chain, 5'- AACUAUUGCUUCAGCUCCAdTdT- 3'
(SEQ ID No. 23)

Antisense, 5' - UGGAGCUGAAGCAAUAGUUdTdT-3' (S
EQ ID No. 33).VEGFR2-21-h2#:

Sense chain, 5'- GGUCCAUUUCAAAUCUCAAdTdT -3'
(SEQ ID No. 61)

Antisense, 5' - UUGAGAUUUGAAAUGGACCdTdT -3' (
SEQ ID No. 104).

Another example is the pair designated as hmTF-21-hm6# and hmVR2-21-h1#, which have the following sequences:

TF1hm6#:

Sense chain, 5'- CGGCAGCUGUACAUUGACUdTdT - 3'
(SEQ ID No. 26)

Antisense, 5' - AGUCAAUGUACAGCUGCCGdTdT -3' (
SEQ ID No. 36).VEGFR2-21-h1#:

Sense chain, 5'- GCCUAGUGUUUCUCUUGAUdTdT-3' (
SEQ ID No. 60)

Antisense, 5' - AUCAAGAGAAACACUAGGCdTdT -3' (
SEQ ID No. 103).

In still another example, the siRNA molecules are the pair designated as hmTF-21-hm6# and hmVR2-21-h2#, which have the following sequences:

TF1hm6#:

Sense chain, 5'- CGGCAGCUGUACAUUGACUdTdT - 3'
(SEQ ID No. 26)

Antisense, 5' - AGUCAAUGUACAGCUGCCGdTdT -3' (
SEQ ID No. 36).VEGFR2-21-h2#:

Sense chain, 5'- GGUCCAUUUCAAAUCUCAAdTdT -3'
(SEQ ID No. 61)

Antisense, 5' - UUGAGAUUUGAAAUGGACCdTdT -3' (
SEQ ID No. 104).

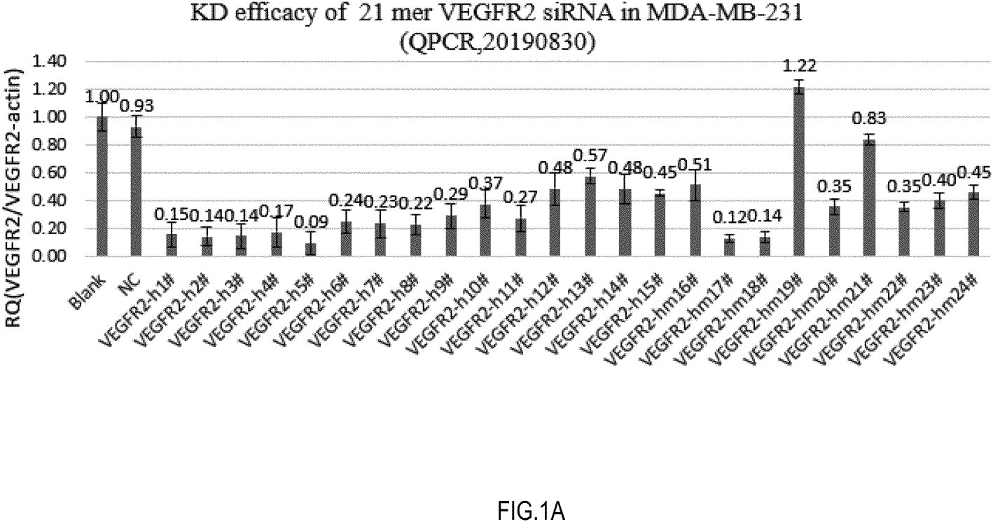

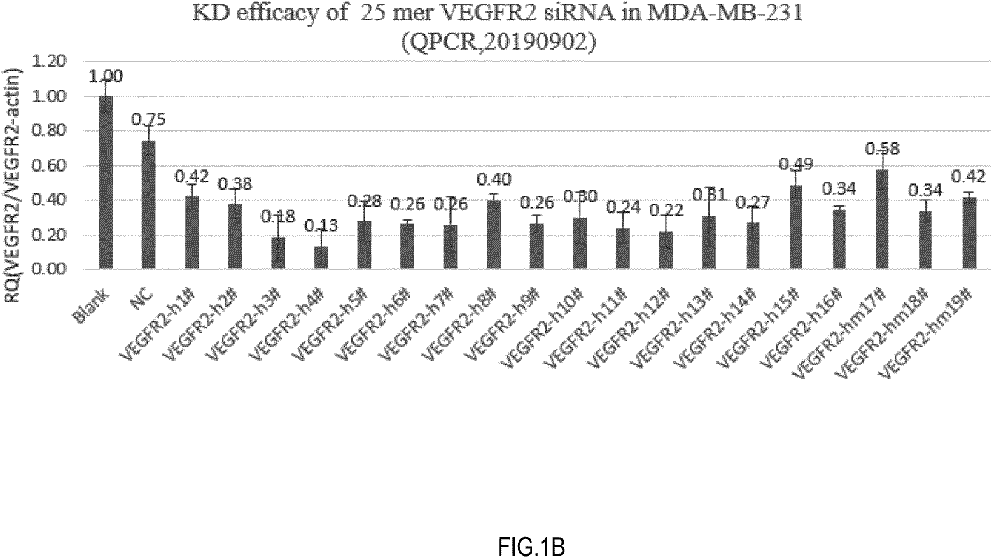

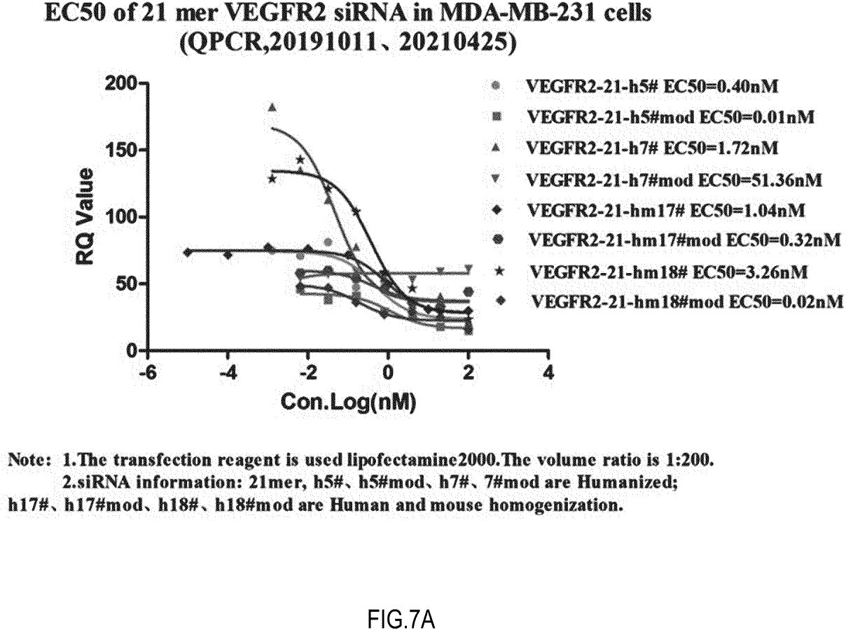

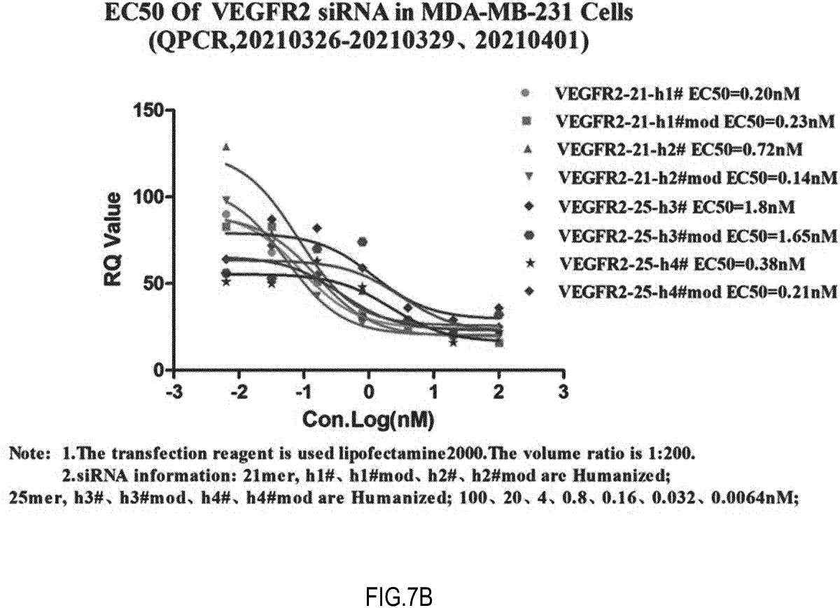

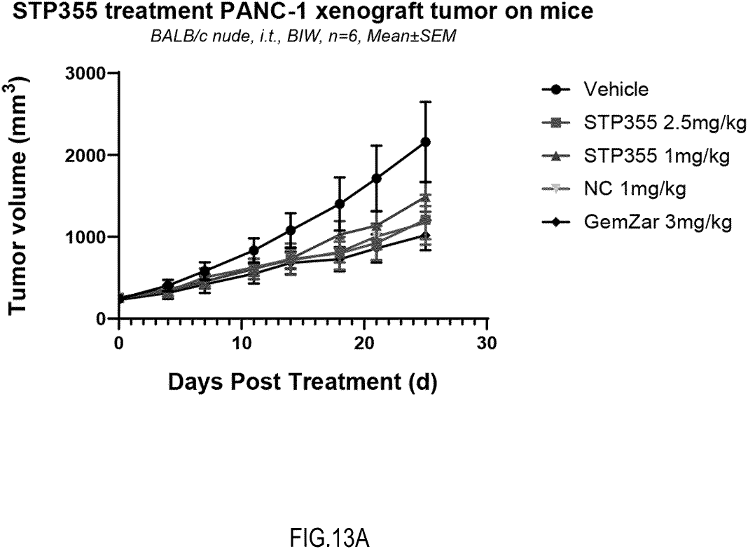

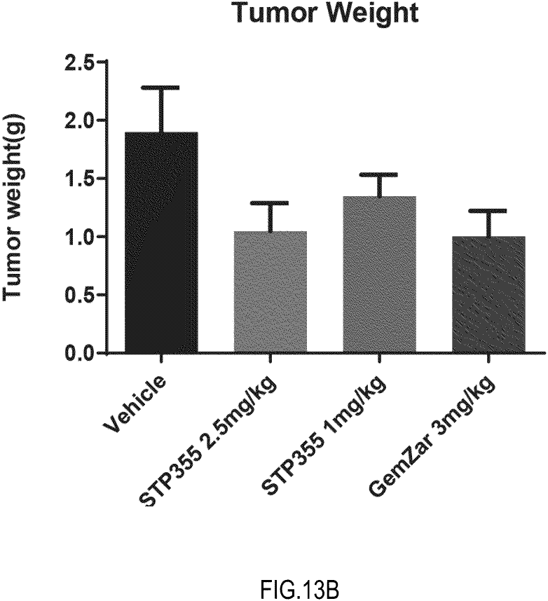

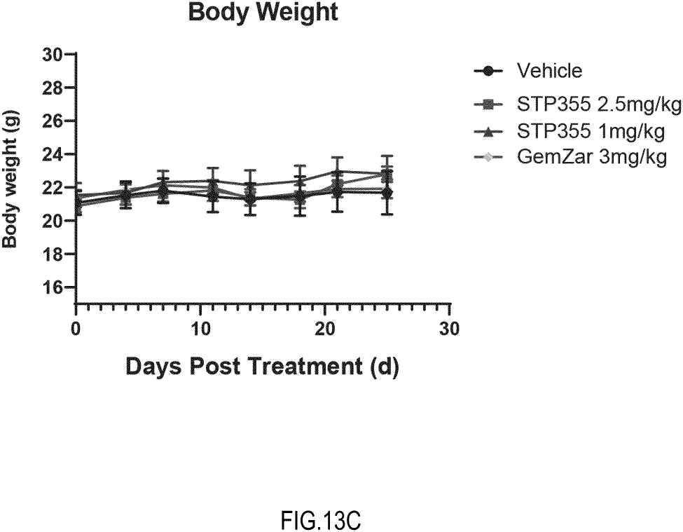

Suitable siRNA molecules have the desired activity may be identified using a method involving the steps of: (a) creating a collection of siRNA molecules designed to target a complementary nucleotide sequence in the target mRNA molecules, wherein the targeting strands of the siRNA molecules comprise various sequences of nucleotides; (b) selecting the siRNA molecules that show the highest desired effect against the target mRNA molecules in vitro including primary screening (FIGS. 1A-1B) and EC₅₀ assays (FIGS. 7-9); (c) evaluating the selected siRNA molecules in an animal tumor models (FIGS. 13-15); and (d) selecting the siRNA molecules that show the greatest efficacy in the model for their silencing activity and therapeutic effect.

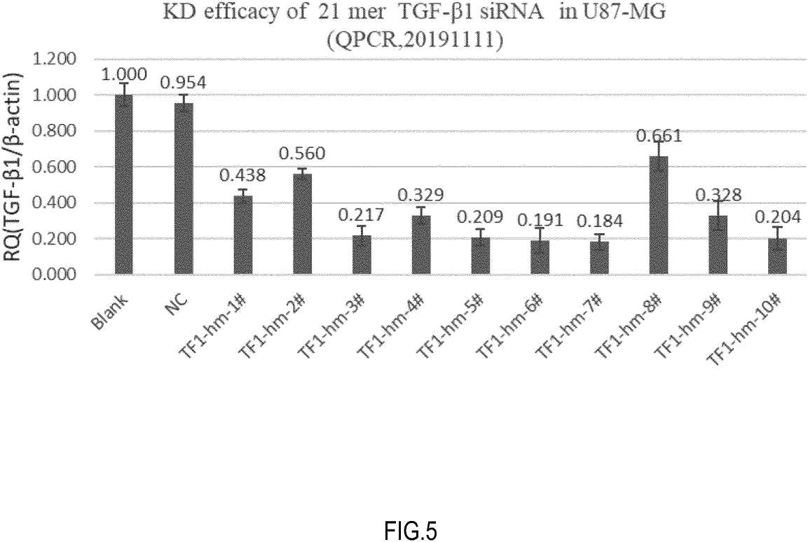

In one embodiment, an animal model for validation of the candidate siRNAs is a xenograft model in a nude mouse. In another embodiment, the animal disease model is an immune competent C57BL/B6 mouse model (FIG. 5). In another embodiment, the method includes the steps of adding a pharmaceutically acceptable carrier to each of the siRNA molecules selected by step (c) to form pharmaceutical compositions and evaluating each of the pharmaceutical compositions in the animal tumor model or models.

The siRNA sequences may be prepared so that each duplex may target and inhibit the same gene of, at least, both human and mouse, or human and nonhuman primate (Tables 1 and 2). In one aspect, the siRNA molecules bind to both a human mRNA molecule and a homologous mouse mRNA molecule. That is, the human and mouse mRNA molecules encode proteins that are substantially the same in structure or function. Therefore, the efficacy and toxicity reactions observed in the mouse disease models predict what will happen in humans. siRNA molecules tested in a mouse model are expected to be good candidates of pharmaceutical agents for use in human.

In one embodiment, the siRNA molecules are selected from those identified in Table 1 and can bind to and induce degradation of TGFβ1 mRNA and VEGFR2 mRNA simultaneously in a mammalian cell or tissue.

The siRNA molecules are combined with a pharmaceutically acceptable carrier to provide pharmaceutical compositions for administering to a mammal. In one aspect of this embodiment, the mammal is a laboratory animal, which includes dogs, cats, pigs, non-human primates, and rodents, such as mice, rats, and guinea pigs. In another aspect, the mammal is a human.

The carrier is a histidine-lysine copolymer that forms a nanoparticle containing an siRNA molecule. One aspect of this embodiment, the carrier is selected from the group consisting of the HKP species, H3K4b, H3K(+H)4b and HK-RCOOH in the HKP series, which have a Lysine backbone or a RCOOH scaffold with four or three branches containing multiple repeats of Histidine, Lysine, or Asparagine. When an aqueous solution of an HKP was mixed with siRNA at a N/P ratio of 2.5:1 by mass, the nanoparticles (average size of 40-400 nm in diameter) were self-assembled. In another aspect, the HKP may have the formula: (R)K(R)-K(R)-(R)K(X), where R=KHHHKHHHKHHHKHHHK, or R=KHHHKHHHNHHHNHHHN, X=C(O)NH₂, where K=lysine, H=histidine, and N=asparagine. In another aspect, the HKP may have the formula: (R)K(R)-K(R)-(R)K(X), where R=KHHHKHHHKHHHKHHHK, or R= KHHHKHHHKHHHHKHHHK, X=C(O)NH2, K=lysine, H=histidine. In still another aspect, the HKP may have the formula: (R)-Lys(R)-Lys(R)-Gly-Ala-Pro-Gly-Ala-Pro-Gly-Ala-Pro-Gly-Arg-Gly-Val-Arg-COOH, where R=KHHHKHHHKHHHKHHHK. In still another aspect, the HKP may have the formula: (R)-Lys(R)-Lys(R)-Gly-Ala-Pro-Gly-Ala-Pro-Ala-Pro-Gly-Ala-Pro-Gly-Arg-Arg-Gly-Val-Arg-COOH, where R=KHHHKHHHKHHHKHHHK.

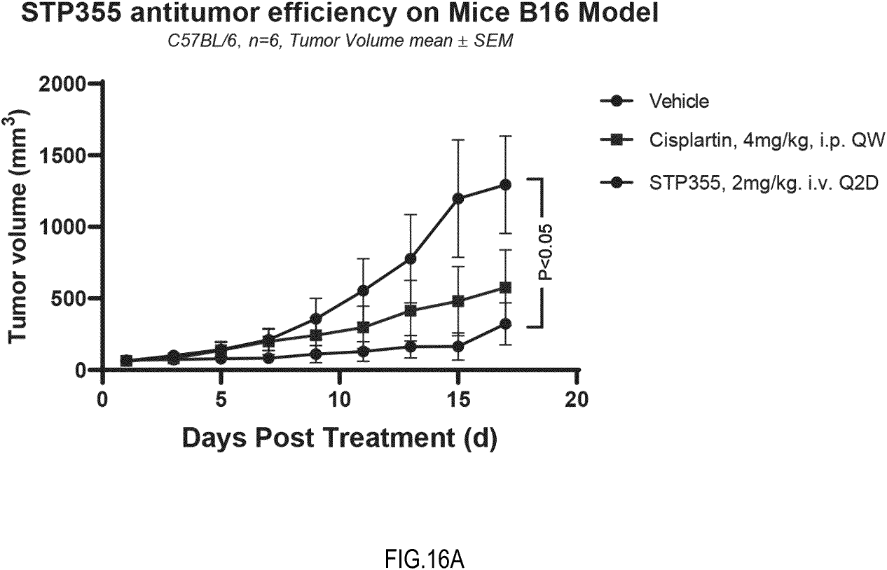

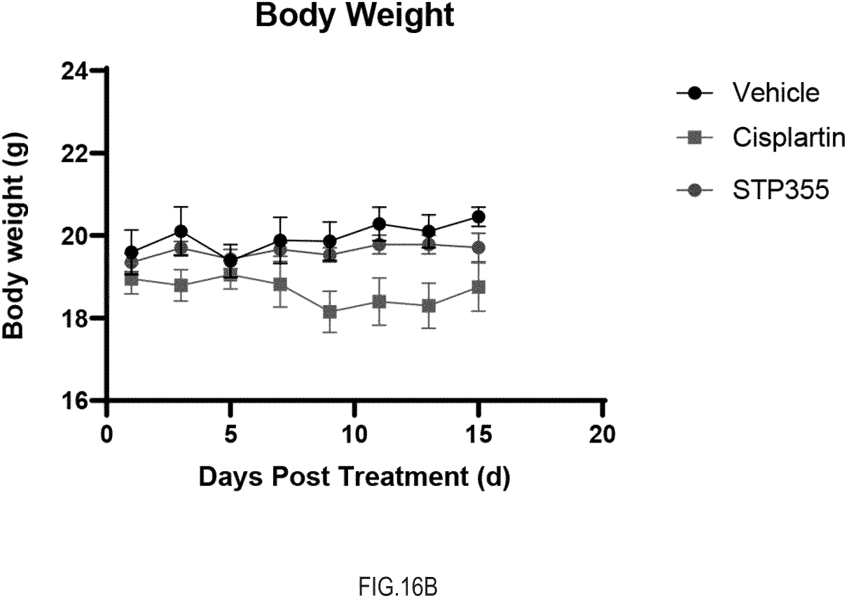

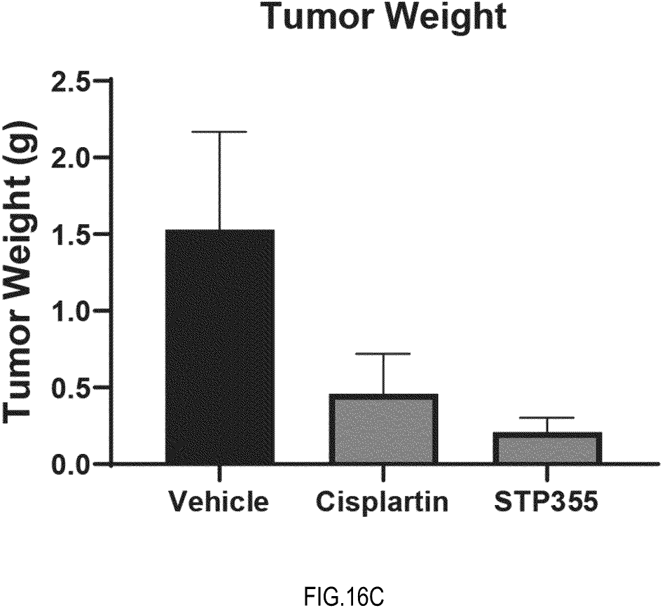

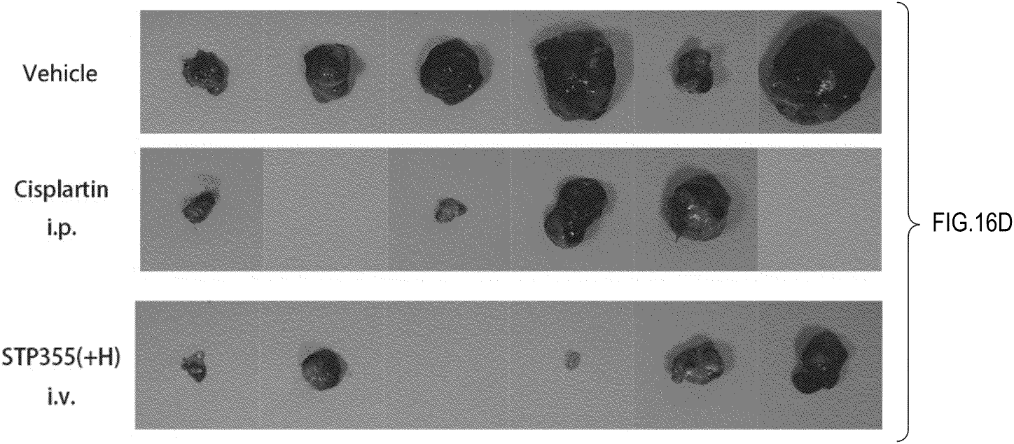

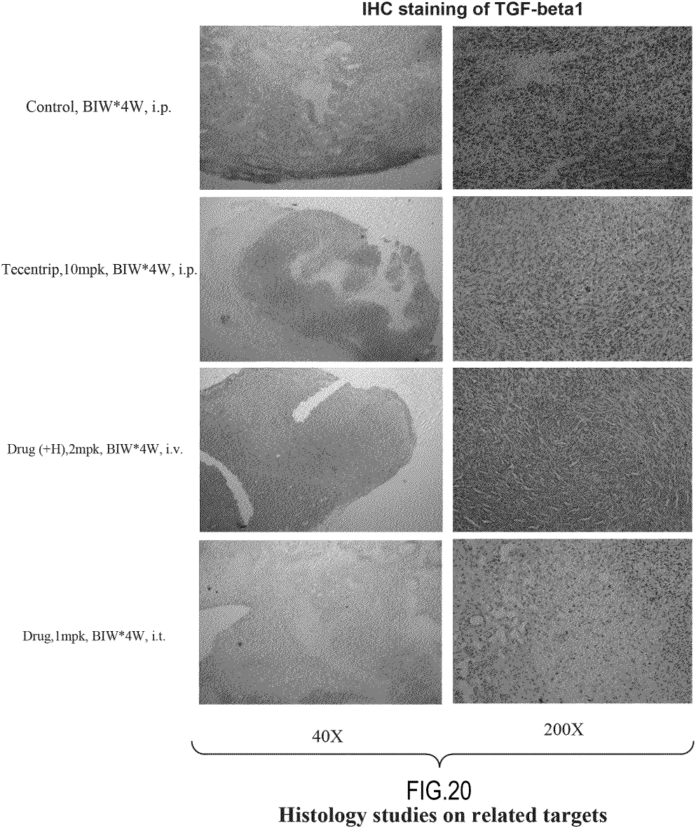

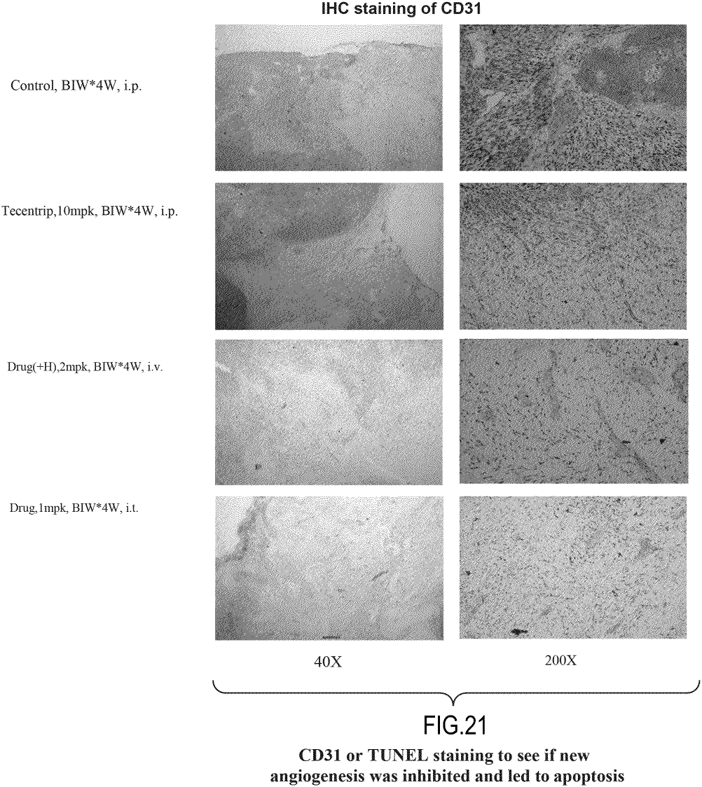

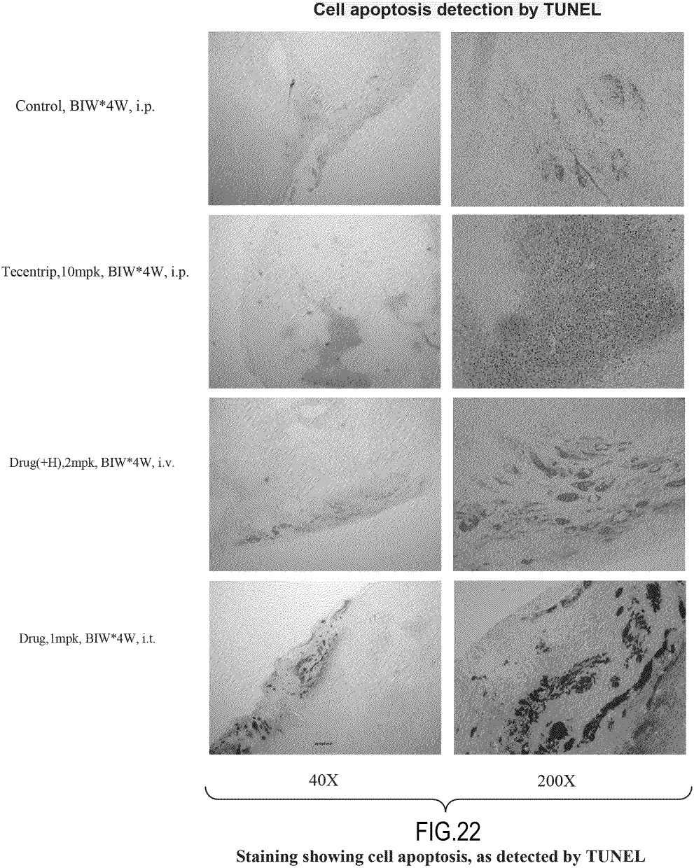

The compositions described herein are useful for simultaneously down-regulating TGF-β1/MAPK signal transduction and the VEGF/VEGFR2 signal pathway in the cells of a tissue of a mammal, as shown in (FIGS. 5A-5D, Histology staining). In some embodiments administration may be into the tumor tissue. In other embodiments, the composition is administered by subcutaneous injection. In still other embodiments, it is administered intravenously or intraperitoneally. In some embodiments, the mammal is a human. A therapeutically effective amount of the composition is administered to the tissue of the mammal in a formulation of HKP-siRNA. Tumor growth was inhibited with the decrease of neo-angiogenesis in the tumor (FIGS. 16 and 20-22, CD31 staining).

### Determination of Efficacy of the siRNA Molecules

Depending on the particular target VEGFR2 and TGFβ1 RNA sequences and the dose of the nanoparticle composition delivered, partial or complete loss of function for the VEGFR2 and TGFβ1 RNAs may be observed. A reduction or loss of RNA levels or expression (either VEGFR2_and TGFβ1 RNA expression or encoded polypeptide expression) in at least 50%, 60%, 70%, 80%, 90%, 95% or 99% or more of targeted cells is exemplary. Inhibition of VEGFR2 and TGFβ1 RNA levels or expression refers to the absence (or observable decrease) in the level of VEGFR2 and_TGFβ1 RNA or VEGFR2 and TGFβ1_RNA-encoded protein. Specificity refers to the ability to inhibit the VEGFR2 and TGFβ1 RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target VEGFR2 and TGFβ1 RNA sequence(s) by the dsRNA agents of the invention also can be measured based upon the effect of administration of such dsRNA agents upon development/progression of a VEGFR2 and TGFβ1-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

### Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds advantageously exhibit high therapeutic indices.

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 µg, or 10, 30, 100, or 1000 mg, or 1-5 g of the compositions can be administered. In general, a suitable dosage unit of the compositions described herein will be in the range of 0.001 to 0.25 mg per kg body weight of the recipient per day, or in the range of 0.01 to 20 µg per kg body weight per day, or in the range of 0.001 to 5 µg per kg of body weight per day, or in the range of 1 to 500 ng per kg of body weight per day, or in the range of 0.01 to 10 µg per kg body weight per day, or in the range of 0.10 to 5 µg per kg body weight per day, or in the range of 0.1 to 2.5 µg per kg body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

Suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

### Treatments

The compositions described herein may be used to treat proliferative diseases, such as cancer, characterized by expression, and particularly altered expression, of VEGFR2 and TGFβ1. Exemplary cancers include liver cancer (e.g. hepatocellular carcinoma or HCC), lung cancer (e.g., NSCLC), colorectal cancer, prostate cancer, pancreatic cancer, ovarian cancer, cervical cancer, brain cancer (e.g., glioblastoma), renal cancer (e.g., papillary renal carcinoma), stomach cancer, esophageal cancer, medulloblastoma, thyroid carcinoma, rhabdomyosarcoma, osteosarcoma, squamous cell carcinoma (e.g., oral squamous cell carcinoma), melanoma, breast cancer, and hematopoietic disorders (e.g., leukemias and lymphomas, and other immune cell-related disorders). Other cancers include bladder, cervical (uterine), endometrial (uterine), head and neck, and oropharyngeal cancers. Advantageously, the cancer is head and neck cancer, bladder cancer, pancreatic cancer, cholangiocarcinoma, lung cancer (NSCLC and SCLC), colon cancer, glioblastoma, breast cancer, gastric adenocarcinomas, prostate cancer, ovarian carcinoma, cervical cancer, AML, ALL, myeloma or non-Hodgkins lymphoma.

The compositions may be administered as described above and, advantageously may be delivered systemically or intratumorally. The compositions may be administered as a monotherapy, i.e. in the absence of another treatment, or may be administered as part of a combination regimen that includes one or more additional medications. Advantageously, when used as part of a combination regimen that includes an effective amount of at least one additional chemotherapy drug. The chemotherapy drug may be, for example, a platinum-containing drug, such as cisplatin, oxaloplatin, or carboplatin.

### EXAMPLES

### Example 1. Selection of Small Nucleic Acids Specific for Human and Mouse TGF-β1 mRNA

Based on proprietary computer-based algorithms, we designed small nucleic acids for TGF- β 1 (Table 1) with the following characteristics: (a) optimal thermodynamic properties; (b) enhanced binding to RISC; (c) immune elimination Activation domain; (d) Human or human-mouse homology; (e) Search sequences through proprietary intellectual property rights; (f) Use blast to try to avoid “off-target effects”; and (g) No interaction when multiple sequences are mixed in a cocktail . The most potent small nucleic acids for each gene were selected by Q RT-PCR (MiiQ, Bio Rad) assay.

TABLE 1

siRNA sequences screened for targeting TGF-β1 mRNA

| siRINA# | SEQ ID | (Sense 5′-3′) | SEQ ID | (Antisense 5′-3′) |
|---|---|---|---|---|
| TF1-20190711-1# | 1 | GCAGAGUACACACAGC AUAdTdT | 2 | UAUGCUGUGUGUACUC UGCdTdT |
| TF1-20190711-2# | 3 | CCAGAAAUACAGCAAC AAUdTdT | 4 | AUUGUUGCUGUAUUUC UGGdTdT |
| TF1-20190711-3# | 5 | GCAACAAUUCCUGGCG AUAdTdT | 6 | UAUCGCCAGGAAUUGU UGCdTdT |
| TF1-20190711-4# | 7 | CCUGUGACAGCAGGGA UAAdTdT | 8 | UUAUCCCUGCUGUCAC AGGdTdT |
| TF1-20190711-5# | 9 | GAGUACACACAGCAUA UAUdTdT | 10 | AUAUAUGCUGUGUGU ACUCdTdT |
| TF1-20190711-6# | 11 | GACUCGCCAGAGUGGU UAUdTdT | 12 | AUAACCACUCUGGCGA GUCdTdT |
| TF1-20190711-7# | 13 | GCGUGCUAAUGGUGGA AACdTdT | 14 | GUUUCCACCAUUAGCA CGCdTdT |
| TF1-20190711-8# | 15 | GCAGGGAUAACACACU GCAdTdT | 16 | UGCAGUGUGUUAUCCC UGCdTdT |
| TF1-20190711-9# | 17 | GGACAUCAACGGGUUC ACUdTdT | 18 | AGUGAACCCGUUGAUG UCCdTdT |
| TF1-20190711-10# | 19 | CCACCAUUCAUGGCAU GAAdTdT | 20 | UUCAUGCCAUGAAUGG UGGdTdT |
| TF1-21-hm1# | 21 | UCGACAUGGAGCUGGU GAAdTdT | 31 | UUCACCAGCUCCAUGU CGAdTdT |
| TF1-21-hm2# | 22 | AUCGACAUGGAGCUGG UGAdTdT | 32 | UCACCAGCUCCAUGUC GAUdTdT |
| TF1-21-hm3# | 23 | AACUAUUGCUUCAGCU CCAdTdT | 33 | UGGAGCUGAAGCAAUA GUUdTdT |
| TF1-21-hm4# | 24 | ACCAACUAUUGCUUCA GCUdTdT | 34 | AGCUGAAGCAAUAGUU GGUdTdT |
| TF1-21-hm5# | 25 | UGCGGCAGCUGUACAU UGAdTdT | 35 | UCAAUGUACAGCUGCC GCAdTdT |
| TF1-21-hm6# | 26 | CGGCAGCUGUACAUUG ACUdTdT | 36 | AGUCAAUGUACAGCUG CCGdTdT |
| TF1-21-hm7# | 27 | GGCAGCUGUACAUUGA CUUdTdT | 37 | AAGUCAAUGUACAGCU GCCdTdT |
| TF1-21-hm8# | 28 | AAGGGCUACCAUGCCA ACUdTdT | 38 | AGUUGGCAUGGUAGCC CUUdTdT |
| TF1-21-hm9# | 29 | AGGGCUACCAUGCCAA CUUdTdT | 39 | AAGUUGGCAUGGUAGC CCUdTdT |
| TF1-21-hm10# | 30 | GGCUACCAUGCCAACU UCUdTdT | 40 | AGAAGUUGGCAUGGU AGCCdTdT |

### Example 2. Selection of Small Nucleic Acids Specific for Human and Mouse VEGFR2 mRNA

Based on proprietary computer-based algorithms, we designed small nucleic acids for VEGFR2 (Table 2) with the following characteristics: a. optimal thermodynamic characteristics; b. enhanced binding to RISC; c. elimination of immune-activating structures domain; d. with human or human-mouse homology; e. search for sequences through proprietary intellectual property; f. use blast to try to avoid “off-target effects”; g. multiple sequences do not interact when mixed in a cocktail. The most potent small nucleic acids for each gene were selected by Q RT-PCR (MiiQ, Bio Rad) assay.

TABLE 2

siRNA sequences screened for targeting VEGFR2 mRNA

| siRNA# | SEQ ID | (sense 5′-3′) | SEQ ID | (antisense 5′-3′) |
|---|---|---|---|---|
| VEGFR2-25-h1# | 41 | CCUCGGUCAUUUAUGUCUAUGU UCA | 84 | UGAACAUAGACAUAA AUGACCGAGG |
| VEGFR2-25-h2# | 42 | CAGAUCUCCAUUUAUUGCUUCU GUU | 85 | AACAGAAGCAAUAAA UGGAGAUCUG |
| VEGFR2-25-h3# | 43 | GACCAACAUGGAGUCGUGUACA UUA | 86 | UAAUGUACACGACUC CAUGUUGGUC |
| VEGFR2-25-h4# | 44 | CCCUUGAGUCCAAUCACACAAU UAA | 87 | UUAAUUGUGUGAUUG GACUCAAGGG |
| VEGFR2-25-h5# | 45 | GGAGGACUUCCAGGGAGGAAAU AAA | 88 | UUUAUUUCCUCCCUG GAAGUCCUCC |
| VEGFR2-25-h6# | 46 | CAUGGAGCUUAAGAAUGCAUCC UUG | 89 | CAAGGAUGCAUUCUU AAGCUCCAUG |
| VEGFR2-25-h7# | 47 | CCUGGAGAAUCAGACGACAAGU AUU | 90 | AAUACUUGUCGUCUG AUUCUCCAGG |
| VEGFR2-25-h8# | 48 | CCAUGUUCUUCUGGCUACUUCU UGU | 91 | ACAAGAAGUAGCCAG AAGAACAUGG |
| VEGFR2-25-h9# | 49 | GAACUGAAGACAGGCUACUUGU CCA | 92 | UGGACAAGUAGCCUG UCUUCAGUUC |
| VEGFR2-25-h10# | 50 | CCAAGUGAUUGAAGCAGAUGCC UUU | 93 | AAAGGCAUCUGCUUC AAUCACUUGG |
| VEGFR2-25-h11# | 51 | UCAUUCAUAUUGGUCACCAUCU CAA | 94 | UUGAGAUGGUGACCA AUAUGAAUGA |
| VEGFR2-25-h12# | 52 | GAGUUCUUGGCAUCGCGAAAGU GUA | 95 | UACACUUUCGCGAUG CCAAGAACUC |
| VEGFR2-25-h13# | 53 | CAGCAGGAAUCAGUCAGUAUCU GCA | 96 | UGCAGAUACUGACUG AUUCCUGCUG |
| VEGFR2-25-h14# | 54 | CAGUGGUAUGGUUCUUGCCUCA GAA | 97 | UUCUGAGGCAAGAAC CAUACCACUG |
| VEGFR2-25-h15# | 55 | AAGCAGGGAGUCUGUGGCAUCU GAA | 98 | UUCAGAUGCCACAGA CUCCCUGCUU |
| VEGFR2-25-h16# | 56 | CCACACUGAGCUCUCCUCCUGU UUA | 99 | UAAACAGGAGGAGAG CUCAGUGUGG |
| VEGFR2-25-hm17# | 57 | CCUACGGACCGUUAAGCGGGCC AAU | 100 | AUUGGCCCGCUUAAC GGUCCGUAGG |
| VEGFR2-25-hm18# | 58 | GCAUCUCAUCUGUUACAGCUUC CAA | 101 | UUGGAAGCUGUAACA GAUGAGAUGC |
| VEGFR2-25-hm19# | 59 | GCUAAGGGCAUGGAGUUCUUGG CAU | 102 | AUGCCAAGAACUCCA UGCCCUUAGC |
| VEGFR2-21-h1# | 60 | GCCUAGUGUUUCUCUUGAUdTdT | 103 | AUCAAGAGAAACACU AGGCdTdT |
| VEGFR2-21-h2# | 61 | GGUCCAUUUCAAAUCUCAAdTdT | 104 | UUGAGAUUUGAAAUG GACCdTdT |
| VEGFR2-21-h3# | 62 | CCUGAUGGUAACAGAAUUUdTdT | 105 | AAAUUCUGUUACCAU CAGGdTdT |
| VEGFR2-21-h4# | 63 | GGGAAUACCCUUCUUCGAAdTdT | 106 | UUCGAAGAAGGGUAU UCCCdTdT |
| VEGFR2-21-h5# | 64 | GCAUCAGCAUAAGAAACUUdTdT | 107 | AAGUUUCUUAUGCUG AUGCdTdT |
| VEGFR2-21-h6# | 65 | CCAAGAAGAACAGCACAUUdTdT | 108 | AAUGUGCUGUUCUUC UUGGdTdT |
| VEGFR2-21-h7# | 66 | GCUGACAUGUACGGUCUAUdTdT | 109 | AUAGACCGUACAUGU CAGCdTdT |
| VEGFR2-21-h8# | 67 | GCAUCACAUCCACUGGUAUdTdT | 110 | AUACCAGUGGAUGUG AUGCdTdT |
| VEGFR2-21-h9# | 68 | CCCAUACCCUUGUGAAGAAdTdT | 111 | UUCUUCACAAGGGUA UGGGdTdT |
| VEGFR2-21-h10# | 69 | CCUCACAUGGUACAAGCUUdTdT | 112 | AAGCUUGUACCAUGU GAGGdTdT |
| VEGFR2-21-h11# | 70 | CCUUAUGAUGCCAGCAAAUdTdT | 113 | AUUUGCUGGCAUCAU AAGGdTdT |
| VEGFR2-21-h12# | 71 | GGAAUUGACAAGACAGCAAdTdT | 114 | UUGCUGUCUUGUCAA UUCCdTdT |
| VEGFR2-21-h13# | 72 | GCUCCUGAAGAUCUGUAUAdTdT | 115 | UAUACAGAUCUUCAG GAGCdTdT |
| VEGFR2-21-h14# | 73 | GCACGAAAUAUCCUCUUAUdTdT | 116 | AUAAGAGGAUAUUUC GUGCdTdT |
| VEGFR2-21-h15# | 74 | GCCUCCCUUUGAAAUGGAUdTdT | 117 | AUCCAUUUCAAAGGG AGGCdTdT |
| VEGFR2-21-hm16# | 75 | CCUCACCUGUUUCCUGUAUdTdT | 118 | AUACAGGAAACAGGU GAGGdTdT |
| VEGFR2-21-hm17# | 76 | GGACUGGCUUUGGCCCAAUdTdT | 119 | AUUGGGCCAAAGCCA GUCCdTdT |
| VEGFR2-21-hm18# | 77 | GGAAAAAACAAAACUGUAAdTdT | 120 | UUACAGUUUUGUUUU UUCCdTdT |
| VEGFR2-21-hm19# | 78 | GGACCGUUAAGCGGGCCAAdTdT | 121 | UUGGCCCGCUUAACG GUCCdTdT |
| VEGFR2-21-hm20# | 79 | GGGAACUGAAGACAGGCUAdTdT | 122 | UAGCCUGUCUUCAGU UCCCdTdT |
| VEGFR2-21-hm21# | 80 | GGCAUGGAGUUCUUGGCAUdTdT | 123 | AUGCCAAGAACUCCA UGCCdTdT |
| VEGFR2-21-hm22# | 81 | CCUCACCUGUUUCCUGUAUdTdT | 124 | AUACAGGAAACAGGU GAGGdTdT |
| VEGFR2-21-hm23# | 82 | CCAAAUUCCAUUAUGACAAdTdT | 125 | UUGUCAUAAUGGAAU UUGGdTdT |
| VEGFR2-21-hm24# | 83 | GCCCCAGGAAGGAAAUGAUdTdT | 126 | AUCAUUUCCUUCCUG GGGCdTdT |
| VEGFR2-21-hm25# | 127 | AAGCUCAGCACACAGAAAGAC | 128 | GUCUUUCUGUGUGCU GAGCUU |
| VEGFR2-21-hm26# | 129 | AAUGCGGCGGUGGUGACAGUA | 130 | UACUGUCACCACCGC CGCAUU |

### Example 3. Preliminary Screening of in Vitro Effects of Small Nucleic Acids With a Length of 25 Nucleotides (25mer) and 21 Nucleotides (21mer) (at the Cellular Level, the Detection Target is VEGFR2)

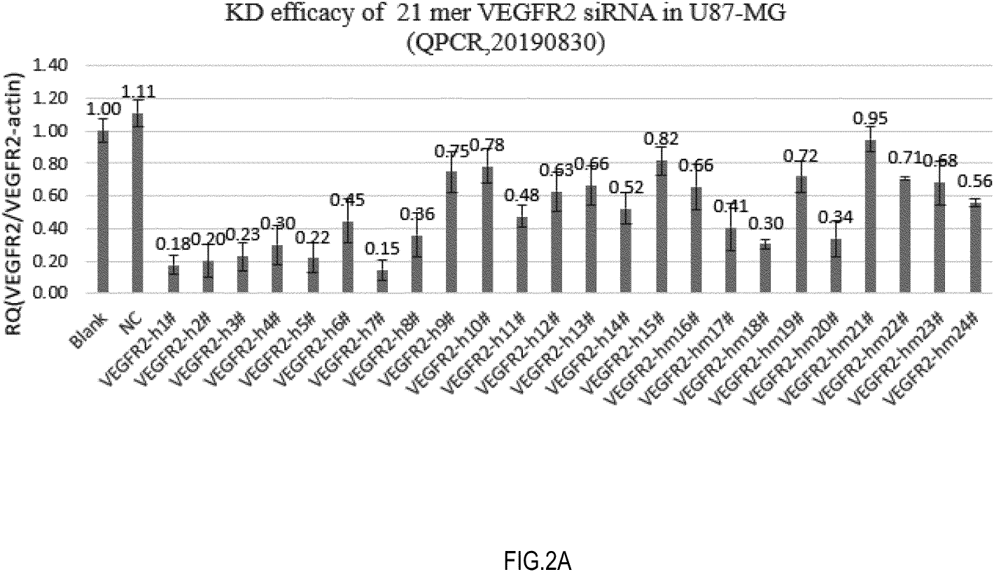

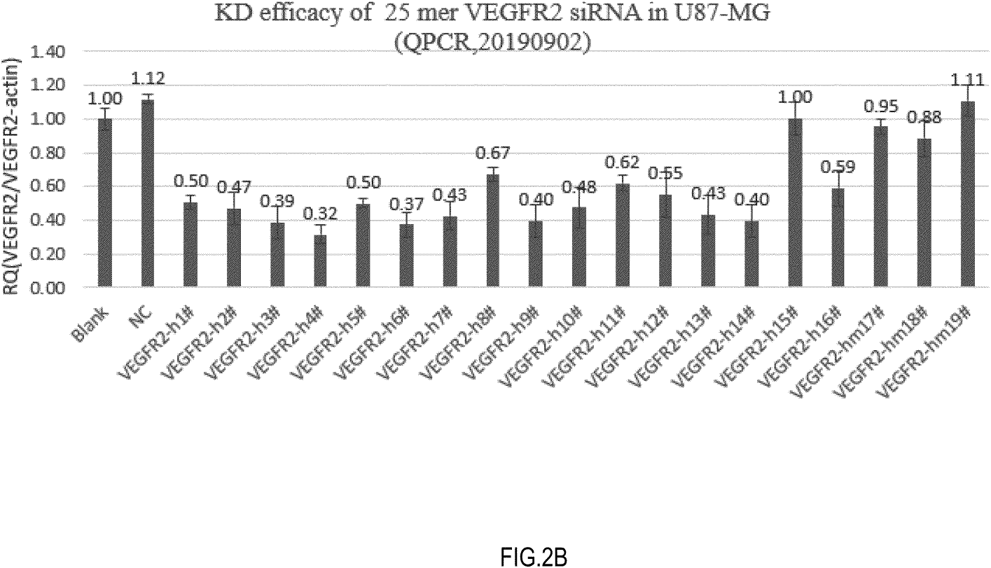

The cell lines used to screen the most efficient small nucleic acids should be those capable of expressing the target gene. In this example, human MDA-MB-231 cells (FIG. 1) and human U87 cells (FIG. 2) were used to screen VEGFR2-specific small nucleic acids.

MDA-MB-231 and U87 cells were seeded in 24-well cell plates (1 × 10⁵/well), and then siRNAs (21mer or 25mer or negative control) were transfected with commercial transfection reagent (lipo2000), and the siRNA transfection concentration was 100 nM. Meanwhile, untreated cells were set as blank control. Total RNA was extracted from cells 24 h after transfection. Reverse transcription was performed using the kit to obtain cDNA according to the manufacturer’s instructions. Relative levels of target gene VEGFR2 mRNA expression were determined by real-time PCR, normalized to the housekeeping gene β-actin. Gene knockdown effectiveness was expressed as a percentage of the blank control. The results are shown in FIGS. 1 and 2. Based on the results of the preliminary screening in MDA-MB-231 and U87-MG cells, the sequences with knockdown effect of 75% or more were selected for subsequent EC₅₀ data detection and analysis. VEGFR2-21-h1#, VEGFR2-21-h2#, VEGFR2-21-h5#, VEGFR2-21-h7#, VEGFR2-21-hm17#, VEGFR2-21-hm18#, VEGFR2-25-h3#, and VEGFR2- 25-h4# were selected and modified as indicated in Table 3 (SEQ ID Nos. 109-124).

TABLE 3

Modified siRNA sequences targeting VEGFR2

| siRNA # | SEQ ID | Sense strand (SS, 5′-3′) | SEQ ID | Antisense strand (AS, 5′-3′) |
|---|---|---|---|---|
| VEGFR2-21-hl#mod | 131 | mGmCmCmUmAmGfUfGfUmUmUmC mUmCmUmUmGmAmUdTdT | 139 | PmAfUmCmAmAmGmAmGmAmA mAmCmAfCmUmAmGmGmCdTdT |
| VEGFR2-21-h2#mod | 132 | mGmGmUmCmCmAfUfUfUmCmAmA mAmUmCmUmCmAmAdTdT | 140 | PmUfUmGmAmGmAmUmUmUmG mAmAmAfUmGmGmAmCmCdTdT |
| VEGFR2-25-h3#mod | 133 | mGmAmCmCmAmAmCmAmUmGmG mAfGfUfCmGmUmGmUmAmCmAmU mUmA | 141 | PmUfAmAmUmGmUmAmCmAmC mGmAmCfUmCmCmAmUmGmUm UmGmGmUmC |
| VEGFR2-25-h4#mod | 134 | mCmCmCmUmUmGmAmGmUmCmC mAfAfUfCmAmCmAmCmAmAmUmU mAmA | 142 | PmUfUmAmAmUmUmGmUmGmU mGmAmUfUmGmGmAmCmUmCm AmAmGmGmG |
| VEGFR2-21-hm17#mod | 135 | mGmGmAmCmUmGfGfCfUmUmUmG mGmCmCmCmAmAmUdTdT | 143 | PmAfUmUmGmGmGmCmCmAmA mAmGmCfCmAmGmUmCmCdTdT |
| VEGFR2-21-hm18#mod | 136 | mGmGmAmAmAmAfAfAfCmAmAmA mAmCmUmGmUmAmAdTdT | 144 | PmUfUmAmCmAmGmUmUmUmU mGmUmUfUmUmUmUmCmCdTdT |
| VEGFR2-21-h5#mod | 137 | mGmCmAmUmCmAfGfCfAmUmAmA mGmAmAmAmCmUmUdTdT | 145 | PmAfAmGmUmUmUmCmUmUmA mUmGmCfUmGmAmUmGmCdTdT |
| VEGFR2-21-h7#mod | 138 | mGmCmUmGmAmCfAfUfGmUmAmC mGmGmUmCmUmAmUdTdT | 146 | PmAfUmAmGmAmCmCmGmUmA mCmAmUfGmUmCmAmGmCdTdT |

Note: 2′F(f), 2′OME(m); phosphorylation (indicated by P).

### Example 4. Preliminary Screening of in Vitro Effects of Small Nucleic Acids With a Length of 21 Nucleotides (at the Cellular Level, the Detection Target is TGF β1)

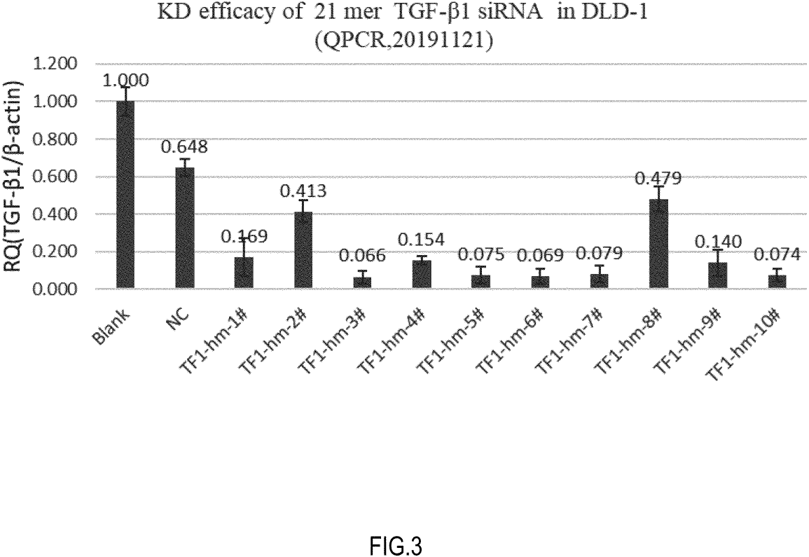

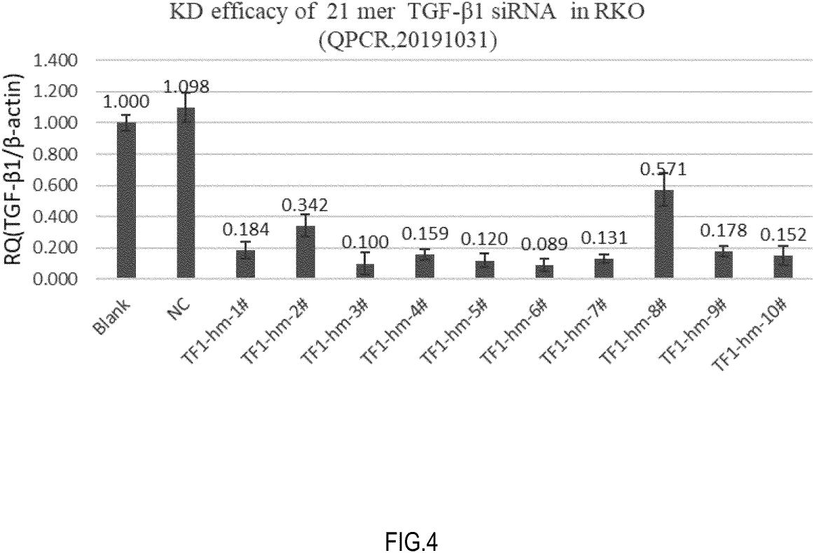

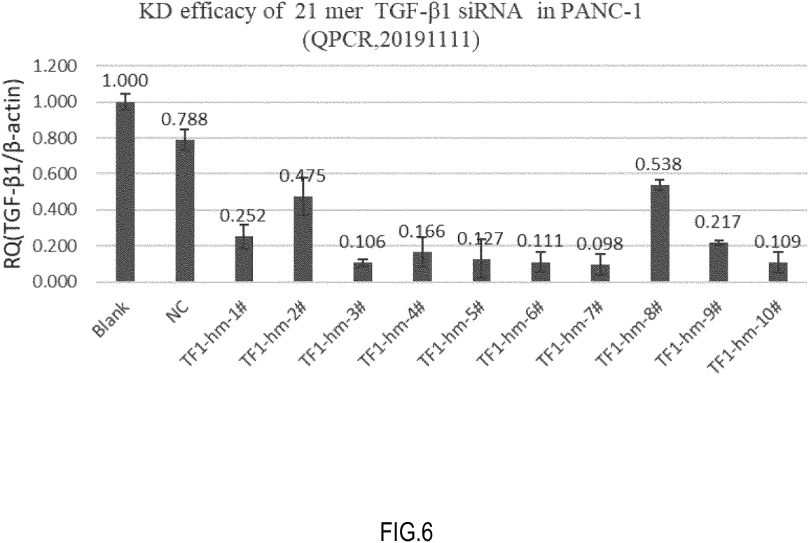

The cell lines used to screen the most efficient small nucleic acids should be those capable of expressing the target gene. In this example, human DLD-1 cells (FIG. 3), human RKO cells (FIG. 4), human U87-MG cells (FIG. 5) and human PANC-1 cells (FIG. 6) were used to screen TGF- β1-specific cells small nucleic acids.

DLD-1, RKO, U87-MG and PANC-1 cells were seeded into 24-well cell plates (1 × 10⁵/well), and then siRNAs (21mer or negative control) were transfected with commercial transfection reagent (lipo2000). The siRNA transfection concentration was 100 nM. Untreated cells were set as a blank control. Total RNA was extracted from cells 24 h after transfection. Reverse transcription was performed using the kit to obtain cDNA according to the manufacturer’s instructions. Relative levels of target gene TGF- β1 mRNA expression were determined by real-time PCR, normalized to the housekeeping gene β-actin. Gene knockdown effectiveness was expressed as a percentage of the blank control. The results are shown in FIGS. 3-6. Based on the preliminary screening results in DLD-1, RKO, U87-MG and PANC-1 cells, the sequences with knockdown effect of 75% or more were selected for subsequent EC50 data detection and analysis. Therefore, TF1-21-hm3# and TF1-21-hm6# were selected as candidate sequences, and these two sequences were modified (see Table 4 for modified sequences).

TABLE 4

Modified siRNA sequences targeting TGF-β1

| siRNA # | SEQ ID | Sense strand (SS, 5′-3′) | SEQ ID | Antisense strand (AS, 5′-3′) |
|---|---|---|---|---|
| TF1-21-hm3#mod | 147 | mAmAmCmUmAmUf UfGfCmUmUmCmA mGmCmUmCmCmAd TdT | 151 | PmUfGmGmAmGmCmUmGm AmAmGmCmAfAmUmAmGm UmUdTdT |
| TF1-21-hm6#mod TF1-20190711 -1#mod TF1-21-hm7#-mod | 148 | mCmGmGmCmAmGf CfUfGmUmAmCmA mUmUmGmAmCmUd TdT | 152 | PmAfGmUmCmAmAmUmGm UmAmCmAmGfCmUmGmCm CmGdTdT |
| | 149 | GCAGAGUACACACA GCAUAdTdT | 153 | PUAUGCUGUGUGUACUCUG CdTdT |
| | 150 | GGCAGCUGUACAU UGACUUdTdT | 154 | PAAGUCAAUGUACAGCUGC CdTdT |

Note: 2′F(f or BOLD FONT), 2′OME(m or in italics); phosphorylation (indicated by P).

### Example 5. Data Comparison Before and After Modification of 25-Nucleotide and 21-Nucleotide siRNA Sequences (at the Cellular Level, the Detection Target is VEGFR2)

The EC₅₀ of candidate sequences before and after modification in different cells (MDA-MB-231, U87-MG and PANC-1) were compared. MDA-MB-231, PANC-1 and U87-MG cells were seeded in 24-well cell plates (1 × 10⁵/well), and siRNA candidates (modified or unmodified) were transfected with multiple concentration gradients in different cells. Use the same procedure as the primary screen. Finally, the data were plotted using the software GraphPad Prism8 and EC₅₀ values were calculated.

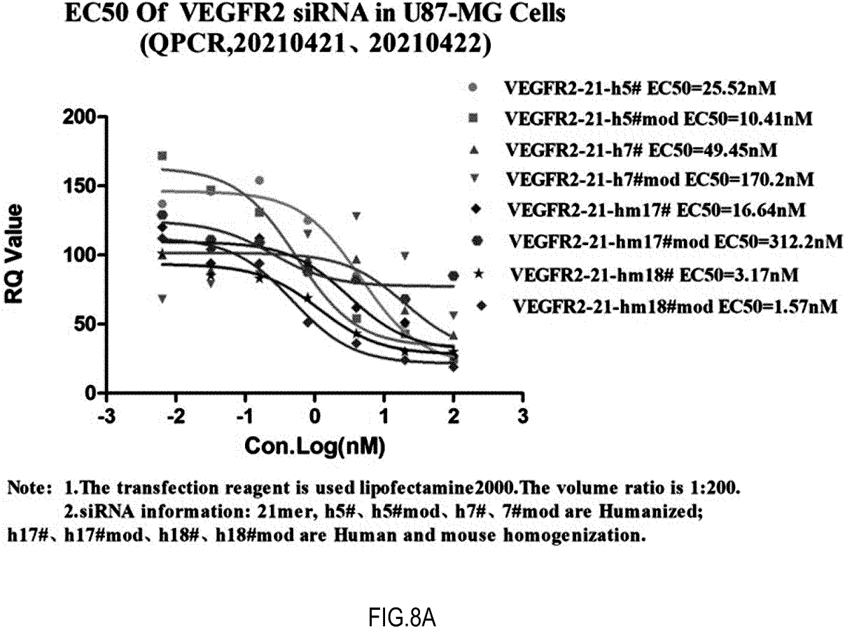

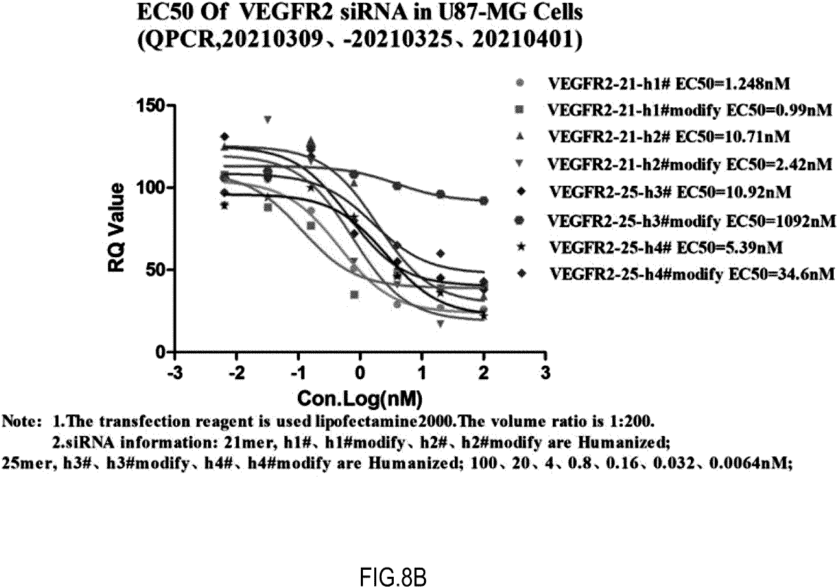

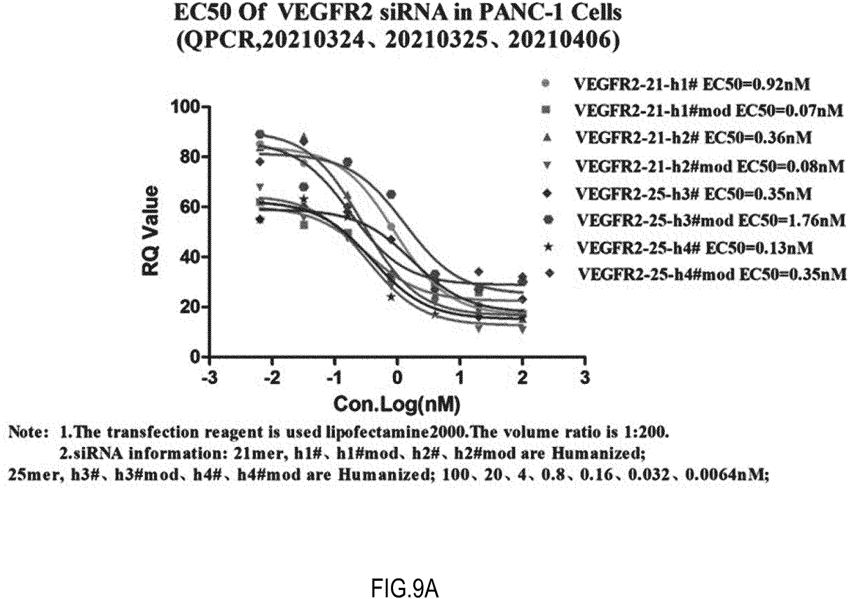

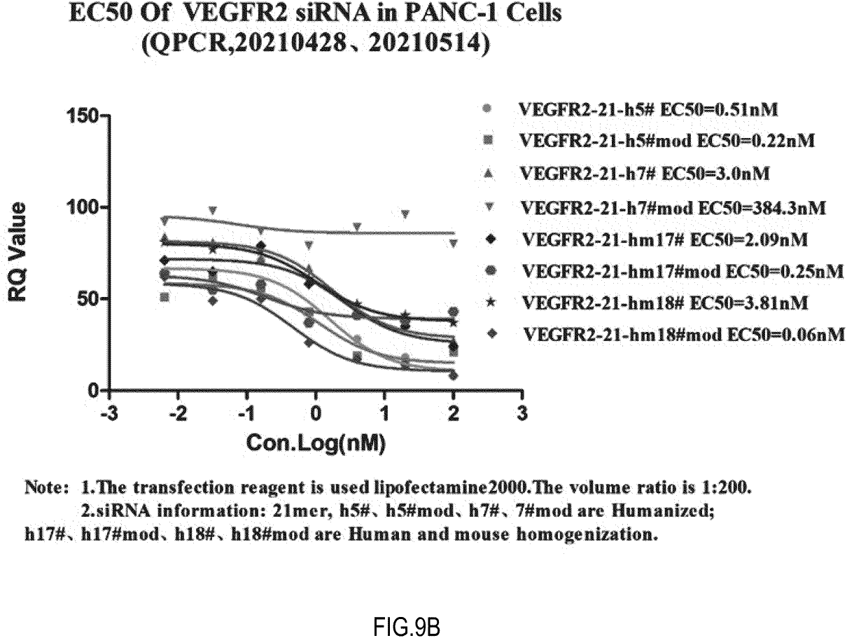

The comparison results of EC₅₀ curves of candidate sequences before and after modification of MDA-MB-231 cells are shown in FIG. 7; the comparison results of EC₅₀ curves of candidate sequences before and after modification of U87-MG cells are shown in FIG. 8; the comparison results of EC₅₀ curves of candidate sequences before and after modification of PANC-1 cells are shown in FIG. 9. Data summary Table 5 shows that each sequence (before and after modification) showed lower EC₅₀ values in PANC-1 and MDA-MB-231, indicating a more significant knockdown effect, and the sequence VEGFR2-21-h5#, VEGFR2-21-h17#, VEGFR2-21-h1#, VEGFR2-21-h2# and VEGFR2-21-hm18# is better than that of unmodified; The EC₅₀ value of candidate VEGFR2 sequences before and after modification in U87-MG is high, and the knockdown effect is not significant, which corresponds to the primary screening results in U87-MG in FIG. 2, but the trend of knockdown effect before and after modification is similar to that in PANC-1 and MDA-MB-231, and the results were consistent.

TABLE 5

EC₅₀ data for siRNA candidates targeting VEGFR2

| | siRNA# | EC₅₀ (nM, 24 h, 24well) | | |
|---|---|---|---|---|
| | | Pancreatic cancer | Brain cancer | Breast cancer |
| ss SEQ ID No. | | PANC-1 | U87-MG | MD-MB-231 |
| 64 | VEGFR2-21-h5# | 0.51 | 25.52 | 0.4 |
| 137 | VEGFR2-21-h5#mod | 0.22 | 10.41 | 0.01 |
| 66 | VEGFR2-21-h7# | 3.00 | 49.45 | 1.72 |
| 138 | VEGFR2-21-h7#mod | 384.3 | 170.2 | 51.36 |
| 76 | VEGFR2-21-hm17# | 2.09 | 16.64 | 1.04 |
| 135 | VEGFR2-21-hm17#mod | 0.25 | 312.2 | 0.32 |
| 77 | VEGFR2-21-hm18# | 3.81 | 3.17 | 3.26 |
| 136 | VEGFR2-21-hm18# mod | 0.06 | 1.57 | 0.02 |
| 60 | VEGFR2-21-h1# | 0.92 | 1.25 | 0.20 |
| 131 | VEGFR2-21-h1#mod | 0.07 | 0.99 | 0.23 |
| 61 | VEGFR2-21-h2# | 0.36 | 10.71 | 0.72 |
| 132 | VEGFR2-21-h2#mod | 0.08 | 2.42 | 0.14 |
| 43 | VEGFR2-25-h3# | 0.35 | 10.92 | 1.80 |
| 133 | VEGFR2-25-h3#mod | 1.76 | 1092.00 | 1.65 |
| 44 | VEGFR2-25-h4# | 0.13 | 5.39 | 0.38 |
| 134 | VEGFR2-25-h 4#mod | 0.35 | 34.60 | 0.21 |

### Example 6. Data Comparison Before and After Modification of 21-nucleotide siRNA Sequences (at the Cellular Level, the Detection Target is TGF-β1)

The EC50 of candidate sequences before and after modification in different cells (U87-MG, DLD-1, SK-Hep-1, BxPC3, A549, HUCCT, PANC-1 and RKO) were compared. U87-MG, DLD-1, SK-Hep-1, BxPC3, A549, HUCCT, PANC-1 and RKO cells were seeded in 24-well cell plates (1 × 10⁵/well) and transfected with multiple concentration gradients in different cells for siRNA candidates (modified or unmodified), use the same procedure as for primary screening. Finally, the data were plotted using the software GraphPad Prism8 and EC₅₀ values were calculated.

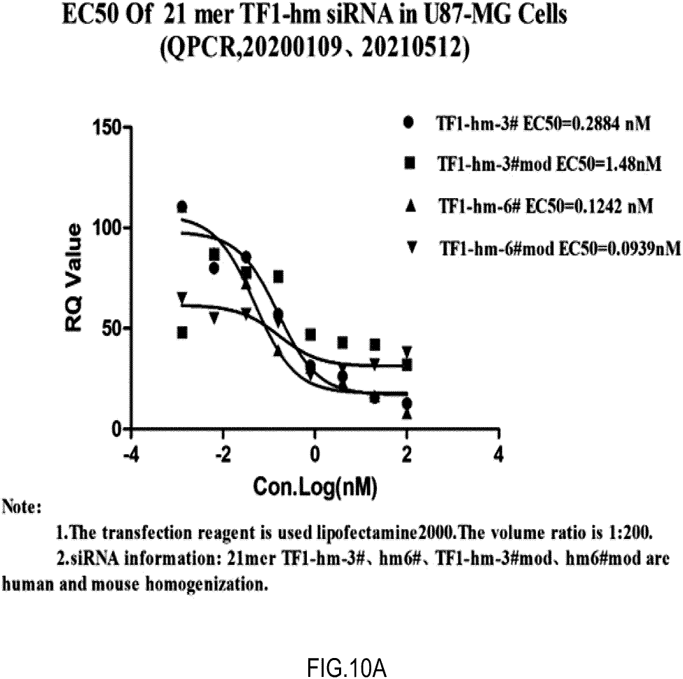

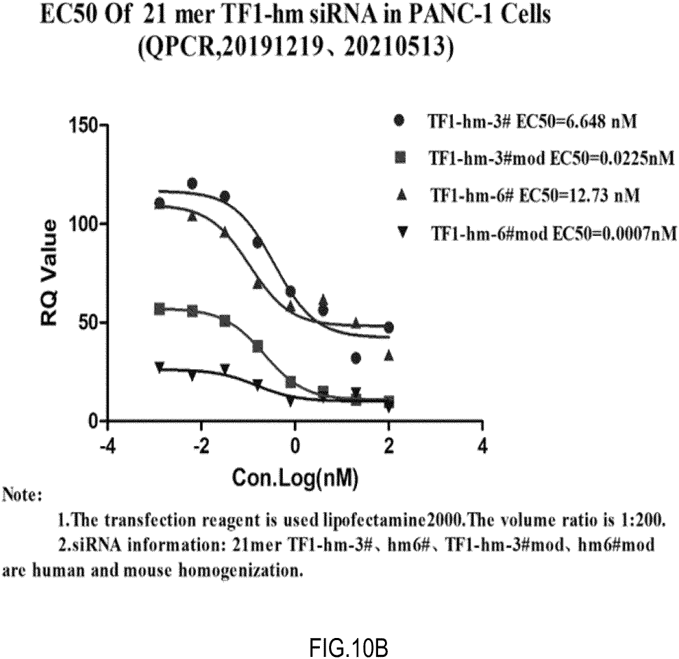

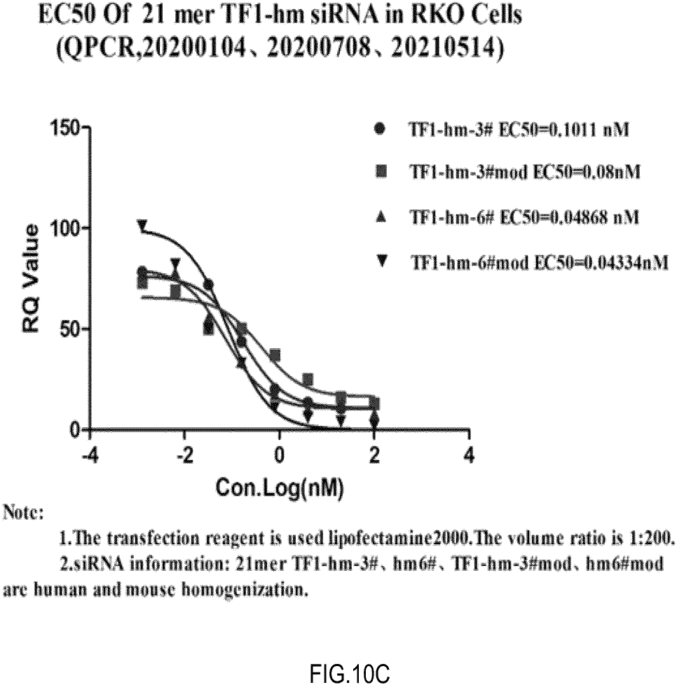

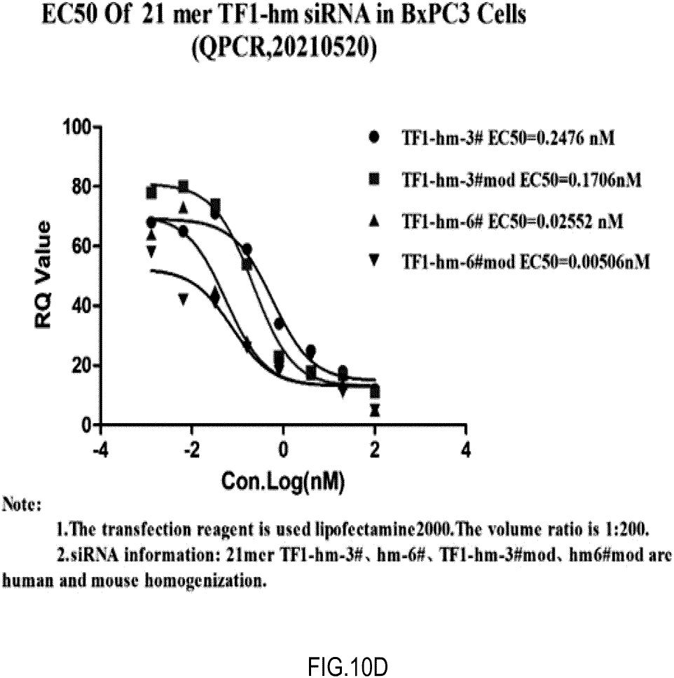

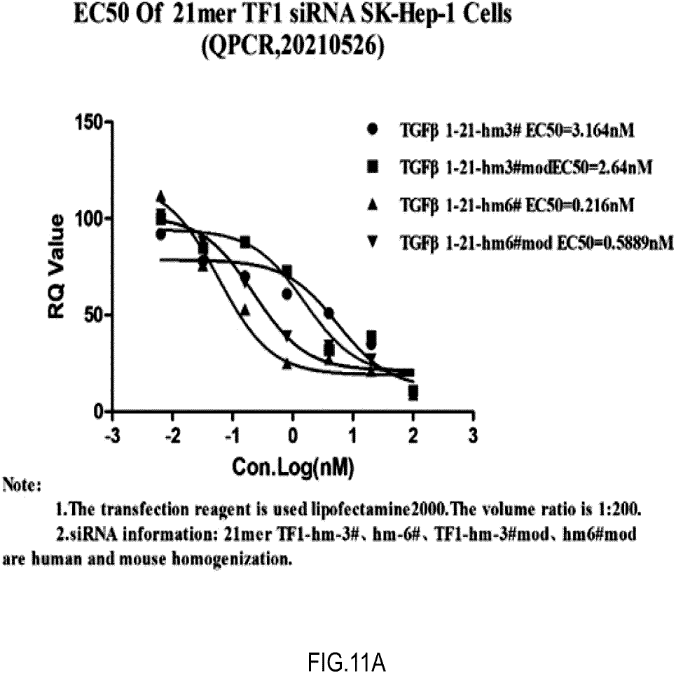

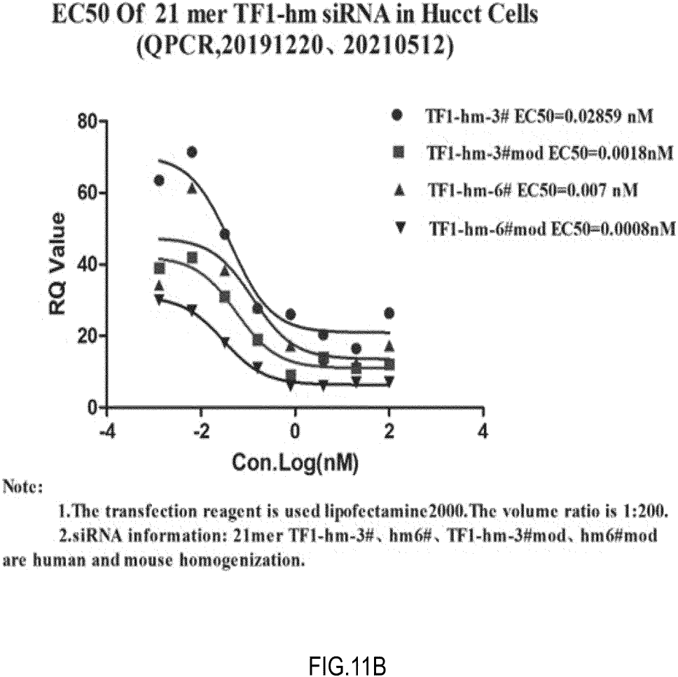

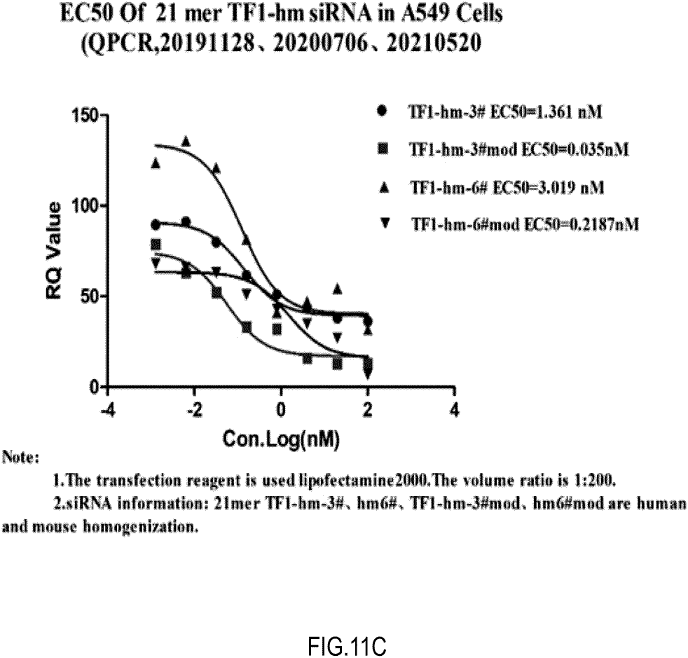

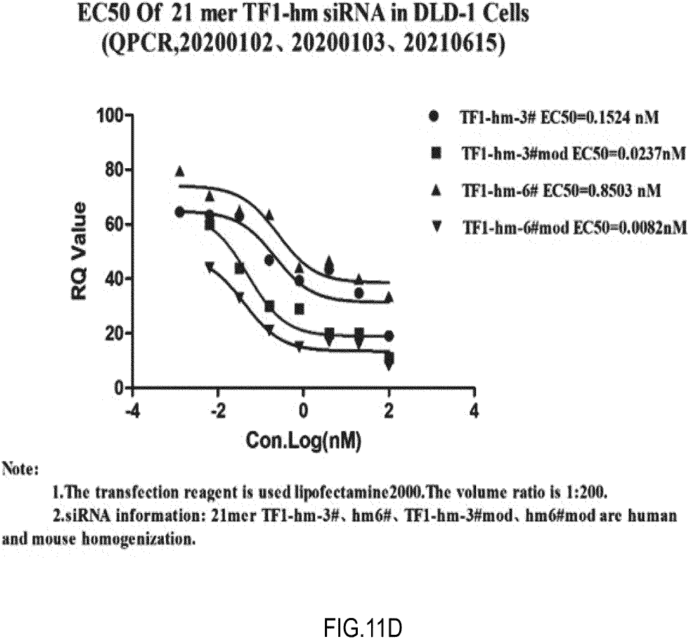

The comparison results of EC₅₀ curves of candidate sequences before and after modification in different cells (U87MG, PANC-1, RKO, BxPC3) are shown in FIG. 10. The EC₅₀ curves of candidate sequences before and after modification in different cells (SK-Hep-1, HUCCT, A549 and DLD-1). The comparison results are shown in FIG. 11, and the data are summarized in Table 6, considering the universality of the candidates (modified or unmodified), selected in a variety of cell lines (U87-MG, DLD-1, SK-Hep-1, BxPC3, A549, HUCCT, PANC-1 and RKO) were used to compare and analyze the EC₅₀ data before and after modification. Each sequence (before and after modification) showed lower EC₅₀ values in U87-MG, DLD-1, SK-Hep-1, BxPC3, A549, HUCCT, PANC-1 and RKO, indicating that it has a more significant Knockdown effect, and showed that the modified knockdown effect was better than that of unmodified (except EC₅₀ values in U87-MG before and after TF1-21-hm3# modification and in SK-Hep-1 before and after TF1-21-hm6# modification the EC₅₀ value).

TABLE 6

EC₅₀ data for siRNA candidates targeting TGF- β1

| | siRNA# | EC50 (nM, 24 h, 24well) | | | | | | | |
|---|---|---|---|---|---|---|---|---|---|
| | | Hucct | SK-Hep1 | A549 | BxPC3 | PANC-1 | DLD-1 | RKO | U87MG |
| ss SEQ ID No | | homo | homo | homo | homo | homo | homo | homo | homo |
| 23 | TF1-21-hm3# | 0.03 | 3.16 | 1.36 | 0.25 | 6.65 | 0.15 | 0.10 | 0.29 |
| 147 | TF1-21-hm3#mod | 0.00 | 2.64 | 0.04 | 0.17 | 0.02 | 0.02 | 0.08 | 1.48 |
| 26 | TF1-21-hm6# | 0.01 | 0.22 | 3.02 | 0.03 | 12.73 | 0.85 | 0.05 | 0.12 |
| 148 | TF1-21-hm6#mod | 0.00 | 0.59 | 0.22 | 0.01 | 0.00 | 0.00 | 0.04 | 0.09 |

TABLE 6A

EC₅₀ data of siRNA candidates targeting TGF-β1

| ss SEQ.ID No. | siRNA# | EC50 (24h, nM, 24well) | | | | | | | | |
|---|---|---|---|---|---|---|---|---|---|---|
| | | Hucct | SK-Hep 1 | A549 | BxPC 3 | PANC -1 | DLD-1 | RKO | U87M G | 293T |
| | | homo | hom o | homo | homo | homo | Homo | homo | homo | homo |
| 1 | TF1-20190711-1# | 0.002 | 1.6 | / | / | / | / | / | / | 0.08 |
| 149 | TF1-20190711-1#mod | / | / | / | / | / | / | / | / | 0.25 |
| 9 | TF1-20190711-5# | / | 9.8 | / | / | / | / | / | / | / |
| 23 | TF1-21-hm3# | 0.029 | / | 1.4 | 11.9 | 6.6 | 0.2 | 0.1 | 0.3 | / |
| 147 | TF1-21-hm3#mod | / | / | 0.04 | / | / | / | 0.08 | / | / |
| 26 | TF1-21-hm6# | 0.007 | / | 3.0 | / | 12.7 | 0.9 | 0.048 | 0.1 | 0.11 |
| 148 | TF1-21-hm6#mod | / | / | / | / | / | / | / | / | 0.1 |
| 27 | TF1-21-hm7# | 0.031 | / | 0.1 | / | 12.0 | 0.7 | 0.1 | 0.6 | / |

### Example 7. The Combined Effect of VEGFR2 siRNA and TGF-β1 siRNA (at Cellular Level, the Detection Targets are TGF-β1 and VEGFR2)

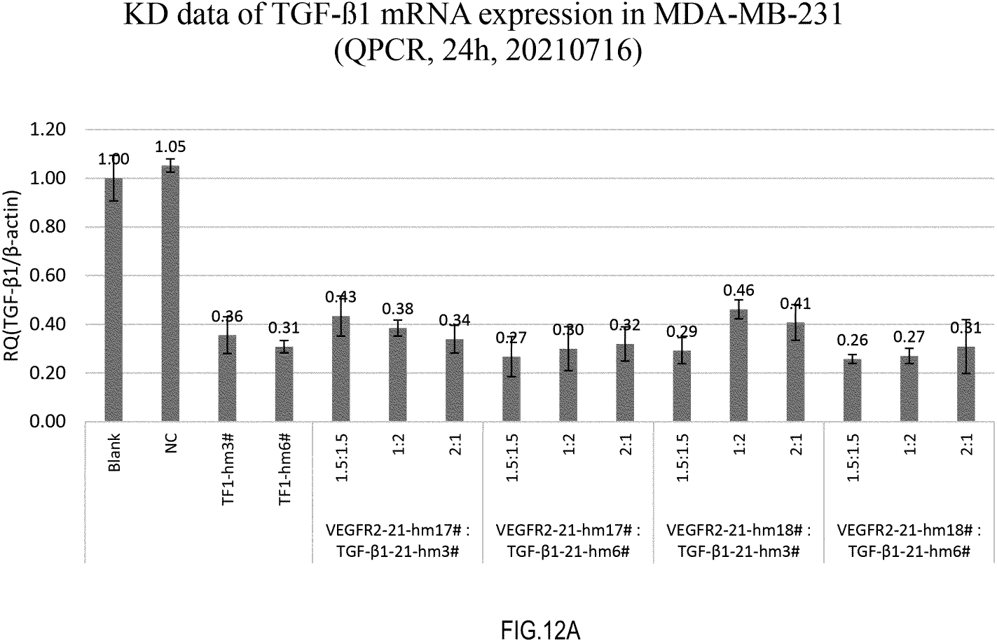

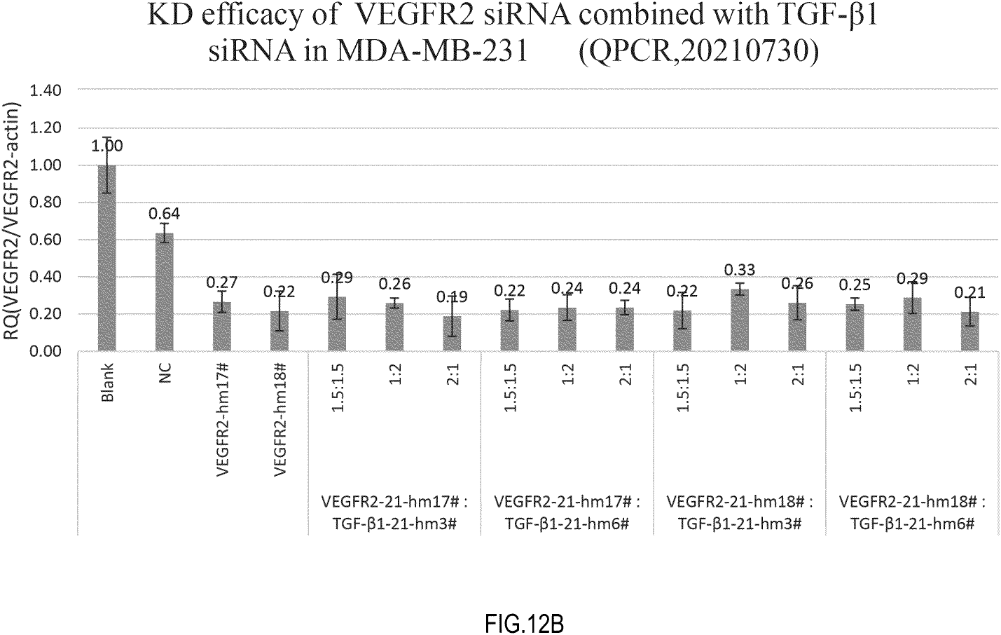

In MDA-MB-231 cells, the knockdown effects of VEGFR2 siRNA and TGF-β1 siRNA were compared at different mass ratios of VEGFR2 siRNA and TGF-β1 siRNA. MDA-MB-231 cells were inoculated into a 24-well cell plate (1 × 10⁵/well), and transfected with different ratios of siRNA (the molecular weight ratio of VEGFR2: TGF-β1 was 1.5:1.5, 1:2, 2:1, respectively). the siRNA transfection concentration was 100 nM, using the same procedure as the primary screening. Finally, the data were plotted using the software GraphPad Prism8 and EC₅₀ values were calculated. The results are shown in FIG. 12. Within the mass ratio of 1:2 to 2:1, the combined effect of VEGFR2 siRNA and TGF-β1 siRNA is similar, and both can achieve a good effect of inhibiting the expression of VEGFR2 and TGF-β1. Considering the convenience of operation, the following example selects a mass ratio of 1:1 as the ratio of the two siRNAs in the STP355 drug.

### Example 8. Selection of Small Nucleic Acid Compositions as Active Components of Drug Candidates

In order to make full use of the novel pharmaceutical preparation mode of combining two small nucleic acids in the present invention and improve the effect of small nucleic acid treatment, the following combination drugs are formulated:

combination 1:

The siRNA molecules are selected from the siRNA molecules determined in Table 1 and Table 2, such as a pair named TF1-21-hm3# and VEGFR2-21-h1# in the table, and their sequences are as follows:

TF1hm3#:

Sense chain: 5'- AACUAUUGCUUCAGCUCCAdTdT-3' (
SEQ ID No. 23)

Antisense: 5' - UGGAGCUGAAGCAAUAGUUdTdT-3' (S
EQ ID No. 33).VEGFR2-21-h1#:

Sense chain: 5'- GCCUAGUGUUUCUCUUGAUdTdT-3' (
SEQ ID No. 60)

Antisense: 5' - AUCAAGAGAAACACUAGGCdTdT-3' (S
EQ ID No. 103).

combination 2:

The siRNA molecules are selected from the siRNA molecules determined in Table 1 and Table 2, such as a pair named TF1-21-hm3# and VEGFR2-21-h2# in the table, and their sequences are as follows:

TF1hm3#:

Sense chain: 5'- AACUAUUGCUUCAGCUCCAdTdT - 3'
(SEQ ID No. 23)

Antisense: 5' - UGGAGCUGAAGCAAUAGUUdTdT - 3'
(SEQ ID No. 33).VEGFR2-21-h2#:

Sense chain: 5'- GGUCCAUUUCAAAUCUCAAdTdT - 3'
(SEQ ID No. 61)

Antisense: 5' - UUGAGAUUUGAAAUGGACCdTdT - 3'
(SEQ ID No. 104).

combination 3:

The siRNA molecules are selected from the siRNA molecules determined in Table 1 and Table 2, such as a pair named TF1-21-hm6# and VEGFR2-21-h1# in the table, and their sequences are as follows:

TF1hm6#:

Sense chain: 5'- CGGCAGCUGUACAUUGACUdTdT - 3'
(SEQ ID No. 26)

Antisense: 5' - AGUCAAUGUACAGCUGCCGdTdT - 3'
(SEQ ID No. 36).VEGFR2-21-h1#:

Sense chain: 5'- GCCUAGUGUUUCUCUUGAUdTdT - 3'
(SEQ ID No. 60)

Antisense: 5' - AUCAAGAGAAACACUAGGCdTdT - 3'
(SEQ ID No. 103).

combination 4:

The siRNA molecules are selected from the siRNA molecules determined in Table 1 and Table 2, such as a pair named TF1-21-hm6# and VEGFR2-21-h2# in the table, and their sequences are as follows:

TF1hm6#:

Sense chain: 5'- CGGCAGCUGUACAUUGACUdTdT - 3'
(SEQ ID No. 26)

Antisense: 5' - AGUCAAUGUACAGCUGCCGdTdT - 3'
(SEQ ID No. 36).VEGFR2-21-h2#:

Sense chain: 5'- GGUCCAUUUCAAAUCUCAAdTdT - 3'
(SEQ ID No. 61)

Antisense: 5' - UUGAGAUUUGAAAUGGACCdTdT - 3'
(SEQ ID No. 104).

combination 5:

The siRNA molecules are selected from the siRNA molecules determined in Table 1 and Table 2, such as a pair named TF1-21-hm3#mod and VEGFR2-21-h1#mod in the table, and their sequences are as follows:

TF1hm3#mod:

Sense chain: 5' - mAmAmCmUmAmUfUfGfCmUmUmCmAm
GmCmUmCmCmAdTdT- 3' (SEQ ID No. 147)

Antisense: 5' -PmUfGmGmAmGmCmUmGmAmAmGmCmAfAm
UmAmGmUmUdTdT -3' (SEQ ID No.151).

VEGFR2h1#mod:

Sense chain: 5' - mGmCmCmUmAmGfUfGfUmUmUmCmUm
CmUmUmGmAmUdTdT- 3' (SEQ ID No. 131)

Antisense: 5' -PmAfUmCmAmAmGmAmGmAmAmAmCmAfCm
UmAmGmGmCdTdT - 3' (SEQ ID No.139).

combination 6:

The siRNA molecules are selected from the siRNA molecules determined in Table 1 and Table 2, such as a pair named TF1-21-hm3#mod and VEGFR2-21-h2#mod in the table, and their sequences are as follows:

TF1hm3#mod:

Sense chain: 5' - mAmAmCmUmAmUfUfGfCmUmUmCmAm
GmCmUmCmCmAdTdT- 3' (SEQ ID No. 147)

Antisense: 5' -PmUfGmGmAmGmCmUmGmAmAmGmCmAfAm
UmAmGmUmUdTdT -3' (SEQ ID No.151).

VEGFR2h2#mod:

Sense chain: 5' - mGmGmUmCmCmAfUfUfUmCmAmAmAm
UmCmUmCmAmAdTdT- 3' (SEQ ID No. 132)

Antisense: 5' - PmUfUmGmAmGmAmUmUmUmGmAmAmAfUmG
mGmAmCmCdTdT -3'(SEQ ID No. 140).

combination 7:

The siRNA molecules are selected from the siRNA molecules determined in Table 1 and Table 2, such as a pair named TF1-21-hm6#mod and VEGFR2-21-h1#mod in the table, and their sequences are as follows:

TF1hm6#mod:

Sense chain: 5'- mCmGmGmCmAmGfCfUfGmUmAmCmAmU
mUmGmAmCmUdTdT- 3' (SEQ ID No. 148)

Antisense: 5' -PmAfGmUmCmAmAmUmGmUmAmCmAmGfCm
UmGmCmCmGdTdT - 3' (SEQ ID No.152).

VEGFR2h1#mod :

Sense chain: 5' - mGmCmCmUmAmGfUfGfUmUmUmCmUm
CmUmUmGmAmUdTdT- 3' (SEQ ID No. 131)

Antisense: 5' - PmAfUmCmAmAmGmAmGmAmAmAmCmAfCmU
mAmGmGmCdTdT -3'(SEQ ID No. 139).

combination 8:

The siRNA molecules are selected from the siRNA molecules determined in Table 1 and Table 2, such as a pair named TF1-21-hm6#mod and VEGFR2-21-h2#mod in the table, and their sequences are as follows:

TF1hm6#mod:

Sense chain: 5'- mCmGmGmCmAmGfCfUfGmUmAmCmAmU
mUmGmAmCmUdTdT- 3' (SEQ ID No. 148)

Antisense: 5' - PmAfGmUmCmAmAmUmGmUmAmCmAmGfCmU
mGmCmCmGdTdT -3'(SEQ ID No. 152).

VEGFR2h2#mod:

Sense chain: 5' - mGmGmUmCmCmAfUfUfUmCmAmAmAm
UmCmUmCmAmAdTdT- 3' (SEQ ID No. 132)

Antisense: 5' -PmUfUmGmAmGmAmUmUmUmGmAmAmAfUm
GmGmAmCmCdTdT - 3'(SEQ ID No.140).

combination 9:

The siRNA molecules are selected from the siRNA molecules determined in Table 1 and Table 2, such as a pair named TF1-21-hm3# and VEGFR2-21-h17# in the table, and their sequences are as follows:

TF1hm3#:

Sense chain: 5'- AACUAUUGCUUCAGCUCCAdTdT - 3'
(SEQ ID No. 23)

Antisense: 5' - UGGAGCUGAAGCAAUAGUUdTdT - 3'
(SEQ ID No. 33).VEGFR2-21-hm17#:

Sense chain: 5'- GGACUGGCUUUGGCCCAAUdTdT - 3'
(SEQ ID No. 76)

Antisense: 5' - AUUGGGCCAAAGCCAGUCCdTdT - 3'
(SEQ ID No. 119).

combination 10:

The siRNA molecules are selected from the siRNA molecules determined in Table 1 and Table 2, such as a pair named TF1-21-hm3# and VEGFR2-21-h18# in the table, and their sequences are as follows:

TF1hm3#:

Sense chain: 5'- AACUAUUGCUUCAGCUCCAdTdT - 3'
(SEQ ID No. 23)

Antisense: 5' - UGGAGCUGAAGCAAUAGUUdTdT - 3'
(SEQ ID No. 33).VEGFR2-21-hm18#:

Sense chain: 5'- GGAAAAAACAAAACUGUAAdTdT - 3'
(SEQ ID No. 77)

Antisense: 5' - UUACAGUUUUGUUUUUUCCdTdT - 3'
(SEQ ID No. 120).

combination 11:

The siRNA molecules are selected from the siRNA molecules determined in Table 1 and Table 2, such as a pair named TF1-21-hm6# and VEGFR2-21-h17# in the table, and their sequences are as follows:

TF1hm6#:

Sense chain: 5'- CGGCAGCUGUACAUUGACUdTdT - 3'
(SEQ ID No. 26)

Antisense: 5' - AGUCAAUGUACAGCUGCCGdTdT - 3'
(SEQ ID No. 36).VEGFR2-21-hm17#:

Sense chain: 5'- GGACUGGCUUUGGCCCAAUdTdT - 3'
(SEQ ID No. 76)

Antisense: 5' -AUUGGGCCAAAGCCAGUCCdTdT - 3' (
SEQ ID No. 119).

combination 12:

The siRNA molecules are selected from the siRNA molecules determined in Table 1 and Table 2, such as a pair named TF1-21-hm6# and VEGFR2-21-h18# in the table, and their sequences are as follows:

TF1hm6#:

Sense chain: 5'- CGGCAGCUGUACAUUGACUdTdT - 3'
(SEQ ID No. 26)

Antisense: 5' - AGUCAAUGUACAGCUGCCGdTdT - 3'
(SEQ ID No. 36).VEGFR2-21-hm18#:

Sense chain: 5'- GGAAAAAACAAAACUGUAAdTdT - 3'
(SEQ ID No. 77)

Antisense: 5' - UUACAGUUUUGUUUUUUCCdTdT - 3'
(SEQ ID No. 120).

combination 13:

The siRNA molecules are selected from the siRNA molecules determined in Table 1 and Table 2, such as a pair named TF1-21-hm3#mod and VEGFR2-21-h17#mod in the table, and their sequences are as follows:

TF1hm3#mod:

Sense chain: 5' - mAmAmCmUmAmUfUfGfCmUmUmCmAm
GmCmUmCmCmAdTdT- 3' (SEQ ID No. 147)

Antisense: 5' -PmUfGmGmAmGmCmUmGmAmAmGmCmAfAm
UmAmGmUmUdTdT -3' (SEQ ID No.151).

VEGFR2hm17#mod:

Sense chain: 5' - mGmGmAmCmUmGfGfCfUmUmUmGmGm
CmCmCmAmAmUdTdT- 3'(SEQ ID No. 135)

Antisense: 5' -PmAfUmUmGmGmGmCmCmAmAmAmGmCfCm
AmGmUmCmCdTdT - 3' (SEQ ID No.143).

combination 14:

The siRNA molecules are selected from the siRNA molecules determined in Table 1 and Table 2, such as a pair named TF1-21-hm3#mod and VEGFR2-21-h18#mod in the table, and their sequences are as follows:

TF1hm3#mod:

Sense chain: 5' - mAmAmCmUmAmUfUfGfCmUmUmCmAm
GmCmUmCmCmAdTdT- 3' (SEQ ID No. 147)

Antisense: 5' -PmUfGmGmAmGmCmUmGmAmAmGmCmAfAm
UmAmGmUmUdTdT -3' (SEQ ID No.151).

VEGFR2hm18#mod:

Sense chain: 5'- mGmGmAmAmAmAfAfAfCmAmAmAmAmC
mUmGmUmAmAdTdT- 3' (SEQ ID No. 136)

Antisense: 5' - PmUfUmAmCmAmGmUmUmUmUmGmUmUfUmU
mUmUmCmCdTdT- 3' (SEQ ID No. 144).

combination 15:

The siRNA molecules are selected from the siRNA molecules determined in Table 1 and Table 2, such as a pair named TF1-21-hm6#mod and VEGFR2-21-h17#mod in the table, and their sequences are as follows:

TF1hm6#mod:

Sense chain: 5'- mCmGmGmCmAmGfCfUfGmUmAmCmAmU
mUmGmAmCmUdTdT- 3' (SEQ ID No. 148)

Antisense: 5' -PmAfGmUmCmAmAmUmGmUmAmCmAmGfCm
UmGmCmCmGdTdT - 3' (SEQ ID No.152).

VEGFR2hm17#mod:

Sense chain: 5' - mGmGmAmCmUmGfGfCfUmUmUmGmGm
CmCmCmAmAmUdTdT- 3' (SEQ ID No. 135)

Antisense: 5' -PmAfUmUmGmGmGmCmCmAmAmAmGmCfCm
AmGmUmCmCdTdT - 3' (SEQ ID No.143).

combination 16:

The siRNA molecules are selected from the siRNA molecules determined in Table 1 and Table 2, such as a pair named TF1-21-hm6#mod and VEGFR2-21-h18#mod in the table, and their sequences are as follows:

TF1hm6#mod:

Sense chain: 5'- mCmGmGmCmAmGfCfUfGmUmAmCmAmU
mUmGmAmCmUdTdT- 3' (SEQ ID No. 148)

Antisense: 5' -PmAfGmUmCmAmAmUmGmUmAmCmAmGfCm
UmGmCmCmGdTdT -3' (SEQ ID No.152).

VEGFR2hm18#mod:

Sense chain: 5'- mGmGmAmAmAmAfAfAfCmAmAmAmAmC
mUmGmUmAmAdTdT- 3' (SEQ ID No. 136)

Antisense: 5' -PmUfUmAmCmAmGmUmUmUmUmGmUmUfUm
UmUmUmCmCdTdT - 3' (SEQ ID No.144).

### Example 9. The Preparation of STP355 Medicine

Select TGF-β1 siRNA and VEGFR2 siRNA as composition 9 (TF-21-hm3# and VEGFR2-21-hm17#) in Example 8, mix them into a solution according to the ratio of 1:1 (mass ratio), and then mix with Polypeptide carriers (HKP or HKP(+H)) form stable nanoparticle formulations.

### Example 10. In Vivo Pharmacodynamics of STP355 in Mouse Pancreatic Cancer (PANC-1) Xenograft Model

The STP355 drug used in this example is the STP355 drug prepared in Example 9. BALB/c nude mice were subcutaneously inoculated with human pancreatic cancer PANC-1 cells on the back, each 4×10⁶/0.2 ml. When the tumor volume grew to about 200 mm³, the tumors were collected and cut into small pieces with a diameter of about 2 mm, and inoculated into small pieces. The mice were subcutaneously administered when the average tumor volume reached 100 mm³. STP355 was administered intratumorally at doses of 1 mg/kg and 2.5 mg/kg, twice a week, for a total of 8 administrations. The other group was given gemcitabine (GemZar) (3 mg/kg) as a positive control, intratumorally administered medicine, twice a week, a total of 8 doses. The tumor volume and mouse body weight of the mice were measured regularly, and the tumor weights were collected at the end of the experiment and analyzed by the software GraphPad Prism8. Mean±SE. FIG. 13 shows that STP355 has a certain inhibitory effect on mouse pancreatic cancer, and the dose of 2.5 mg/kg is close to GemZar.

### Example 11. Pharmacodynamic Test of STP355 in Mouse Breast Cancer (MDA-MB-231) Xenograft Model

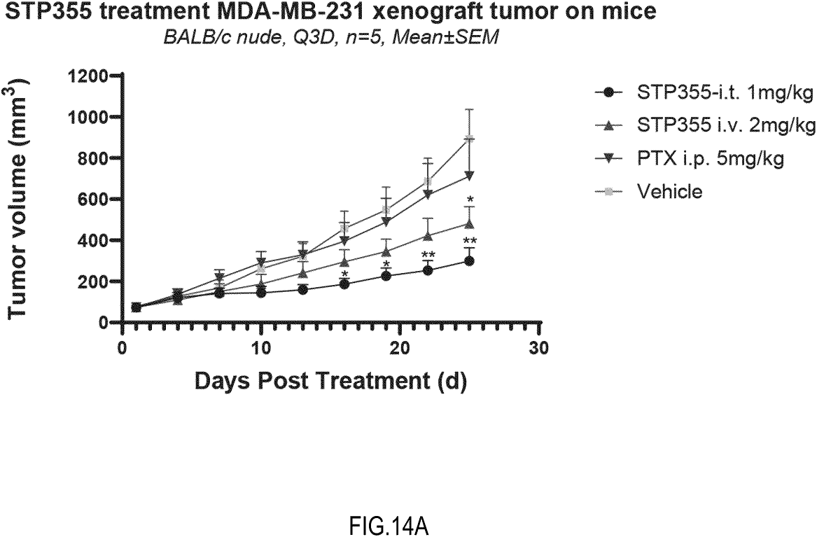

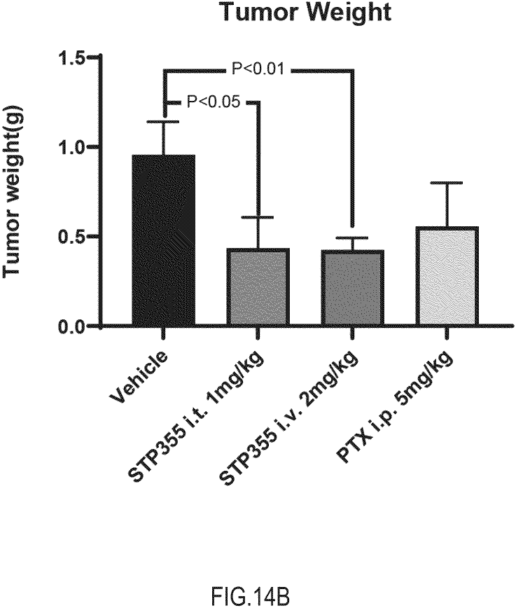

The STP355 drug used in this example is the STP355 drug prepared in Example 9. BALB/c nude mice were subcutaneously inoculated with human breast cancer MDA-MB-231 cells on the back, each 4×10⁶/0.2 ml, and when the average tumor volume reached 100 mm³, group administration was started. STP355 was administered intratumorally at a dose of 1 mg/kg twice a week for a total of 8 doses or intravenously at a dose of 2 mg/kg twice a week for a total of 8 doses. Paclitaxel (PTX) (5 mg/kg) was used as a positive control, intratumorally administered twice a week for a total of 8 administrations. The tumor volume and mouse body weight of the mice were measured regularly, and the tumor weights were collected at the end of the experiment and analyzed by the software GraphPad Prism8. Data points are calculated as mean ± SE. FIG. 14 shows that with STP355 treatment, the inhibitory effect of the two administration methods on mouse breast cancer is better than that of paclitaxel, and the toxicity is much less than that of paclitaxel.

### Example 12. In Vivo Pharmacodynamics of STP355 on Humanized PDL1 Locus Colorectal Cancer Tumors (MC38-hPDL1) in an Immunocompetent Mouse Model

The STP355 drug used in this example is the STP355 drug prepared in Example 9. C57BL/6J mice were subcutaneously inoculated with humanized PDL1 locus colorectal cancer tumor MC38-hPDL1 cells on the back, inoculation volume: 1×10⁶/100 µL/mouse, inoculation location: above the right thigh of the mouse. When the average tumor volume reached 100 mm³, group administration was started, divided into 4 groups, 8 animals/group. In this experiment, the doses of 4 mg/kg and 6 mg/kg were tested, and a model group was established, and the positive control group Tecentrip (atezolizumab) . The drug was administered twice a week for a total of 8 times. On the 0th, 3rd, 7th, 10th, 14th, 17th, 21st, 24th and 28th days, the tumor volume was measured with a vernier caliper, and the tumor growth inhibition rate TGI% was calculated according to the formula.

Calculation formula: TGI % = [1-(Ti-T0)/ (Vi-V0)] ×100%.
Ti represents the mean tumor volume of the treatment group at a certain time point,
T0 represents the mean tumor volume of the treatment group on day 0,
Vi represents the mean tumor volume of the model group at the same time point as Ti,
V0 represents the mean tumor volume of the model group at day 0.

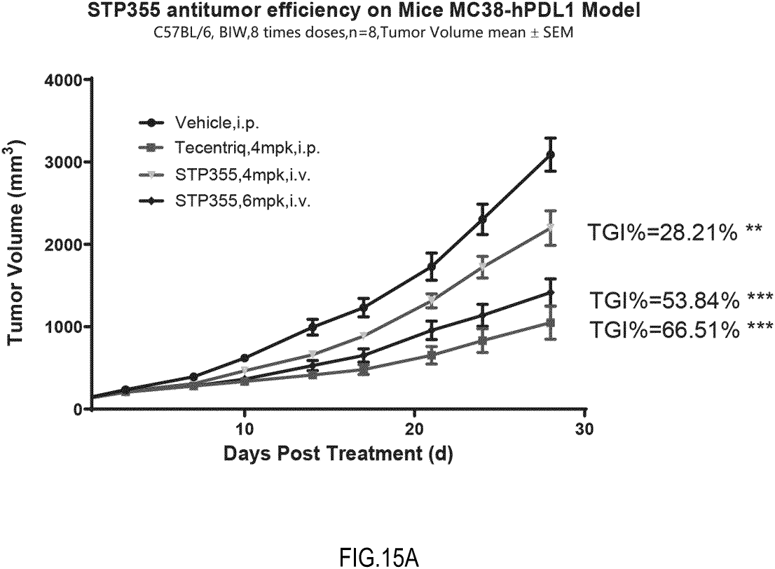

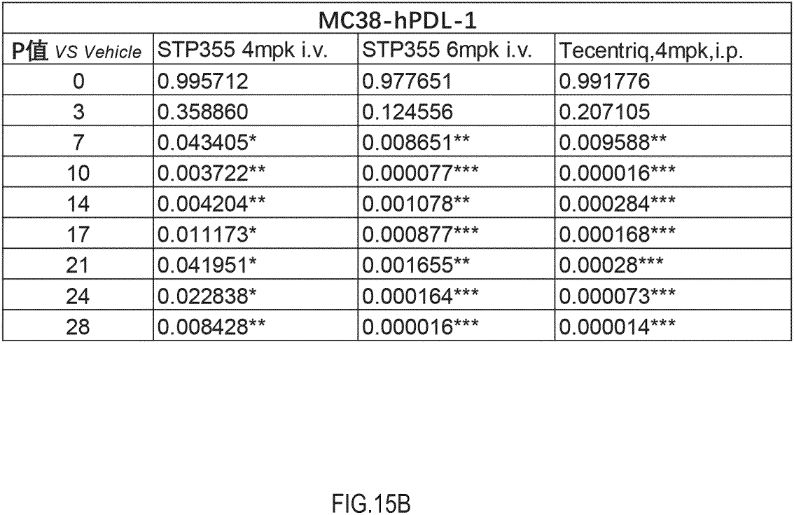

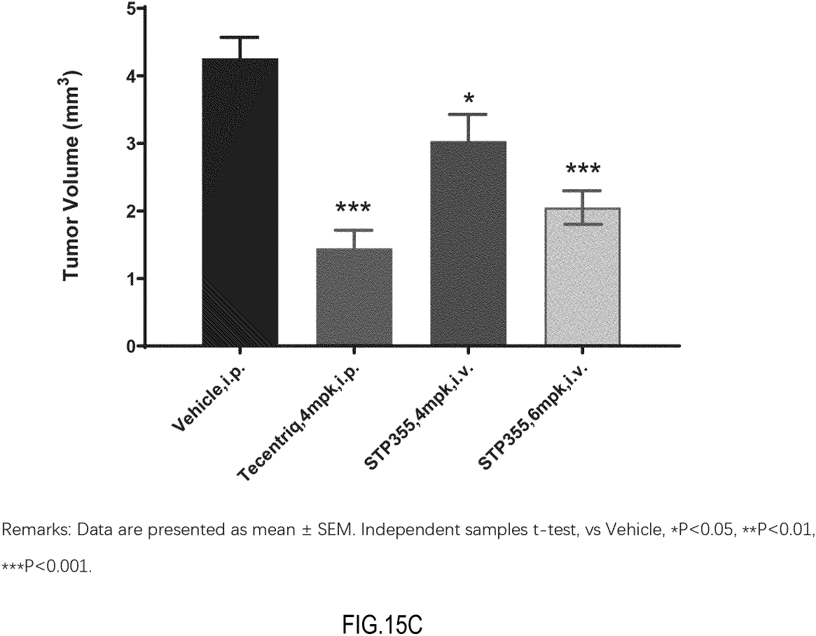

FIG. 15 shows positive control group (Tecentrip, 4mpk, BIW*4W, I.P.), STP355 low dose group (4mpk, BIW*4W, I.V.) and STP355 high dose group (6mpk, BIW*4W, I.V.), from day 7. From the beginning to the 28th day, the tumor volume was significantly smaller than that of the model control group, and the effect of the high-dose STP355 group was comparable to that of the positive control group. From the results of tumor weight, the STP355 high-dose group and the control group were significantly lower than the model group, and the STP355 high-dose group had the same inhibitory effect on tumor growth as Tecentrip.

### Example 13. In Vivo Pharmacodynamics of STP355 on Melanoma (B16) in an Immunocompetent Mouse Model

The STP355 drug used in this example is the STP355 drug prepared in Example 9. C57BL/6J mice were inoculated with B 16-F0 cells subcutaneously on the back, inoculation volume: 1×10⁶/100 µL/mice, 50 mice were inoculated, location: the right back of the mice. The average tumor volume was 80~100 mm³ and started to be administered in groups. The dose of 2 mg/kg was administered intravenously, twice a week, for a total of 8 administrations, and the positive control group was administered with cisplatin, administered intratumorally at a dose of 4 mg/kg, twice a week, A total of 8 doses were administered. FIG. 16 shows that under the current test system, the positive control group (Cisplartin, 4 mg/kg i.p. QW) and the STP355 group (2 mg/kg, 10 µl/g, i.v., Q2D) all showed significant anti-tumor effects, and the STP355 group had better effect and less toxicity.

### Example 14. Pharmacodynamic Comparison Test of Combination Drug and Single Drug in Mouse Breast Cancer (MDA-MB-231) Xenograft Model

Combination 9 (TF1-21-hm3# and VEGFR2-21-hm17#), combination 10 (TF1-21-hm3# and VEGFR2-21-hm18#), combination 11 (TF1-21-hm6# and VEGFR2-21-hm17#), combination 12 (TF1-21-hm6# and VEGFR2-21-hm18#), TGF-β1 siRNA (TF1-21-hm3#), TGF-β1 siRNA (TF1-21-hm6 #), VEGFR2 siRNA (VEGFR2-21-hm17#) and VEGFR2 siRNA (VEGFR2-21-hm18#) were self-assembled with HKP (+H) to form nanoparticles, and the prepared nanoparticles were marked as STP355 (3+17 ), STP355 (3+18), STP355 (6+17), STP355 (6+18), siTGF-β1 (3), siTGF-β1 (6), siVEGF-R2 (17), siVEGF-R2 (18).

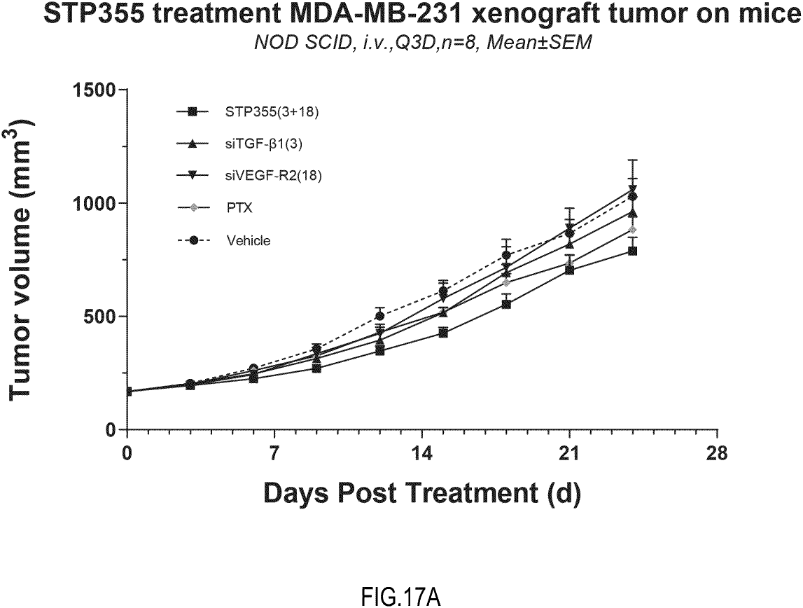

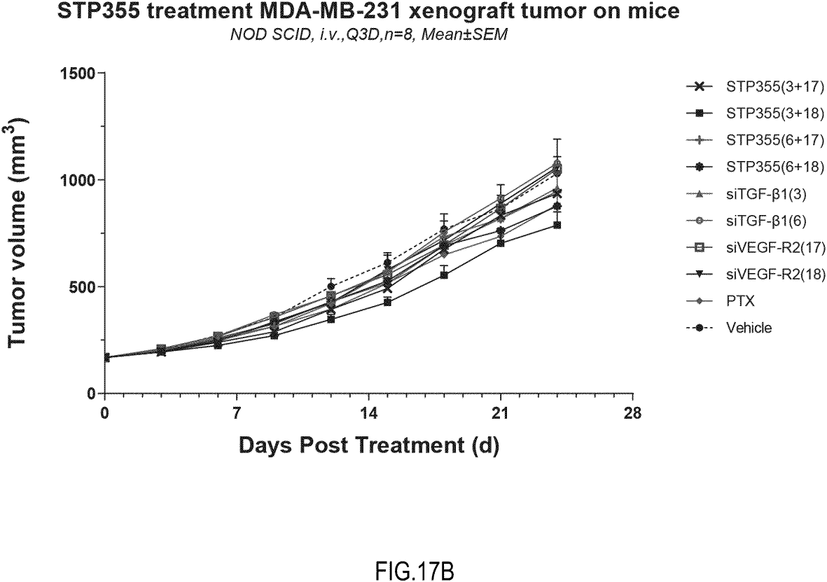

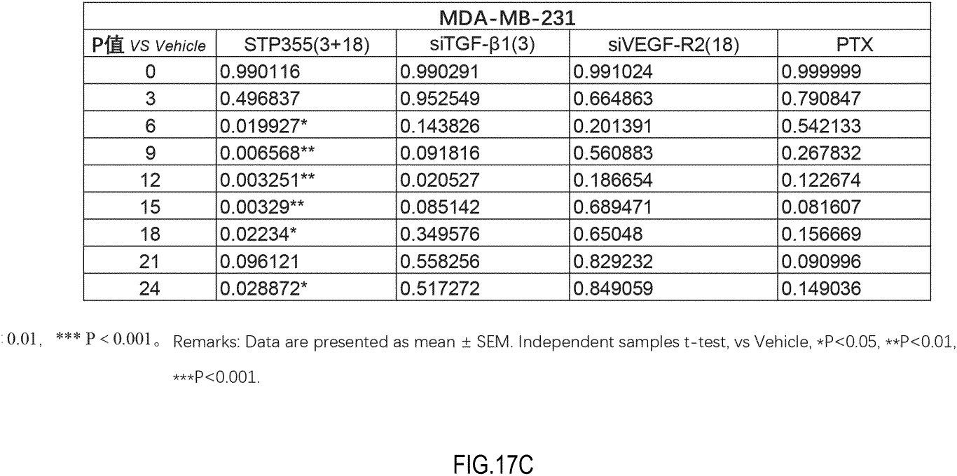

NOD SCID mice were subcutaneously inoculated with human breast cancer MDA-MB-231 cells on the back, each 1×10⁷/0.2 ml. When the average tumor volume reached 100 mm³, the mice were divided into 10 groups, 8 mice/group. The above STP355 drug, TGF-β1 siRNA single drug, and VEGFR2 siRNA single drug were intravenously administered at a dose of 2 mg/kg, once every 3 days, for a total of 8 administrations. Paclitaxel (PTX) (5 mg/kg) was used as a positive control by intraperitoneal injection, once every 3 days, for a total of 8 doses. The tumor volume and mouse body weight of the mice were measured regularly, and the tumor weights were collected at the end of the experiment and analyzed by the software GraphPad Prism9. Data points are calculated as mean ± SE. FIG. 17 shows that compared with the vehicle group, the STP355 (3+18) drug treatment has significantly better inhibitory effect on mouse breast cancer than TGF-β1 siRNA single drug, VEGFR2 siRNA single drug and paclitaxel.

### Example 15. In Vivo Pharmacodynamic Comparison Test of Combination Drug and Modified Drug in Mouse Pancreatic Cancer (PANC-1) Xenograft Model

Combination 9 (TF1-21-hm3# and VEGFR2-21-hm17#), combination 10 (TF1-21-hm3# and VEGFR2-21-hm18#), combination 13 (TF1-21-hm3#mod and VEGFR2-21-hm17#mod), combination 14 (TF1-21-hm3#mod and VEGFR2-21-hm18#mod) were self-assembled with HKP (+H) to form nanoparticles, and the prepared nanoparticles were recorded as For STP355 (3+17), STP355 (3+18), STP355 (3 m+17m), STP355 (3 m+18m).

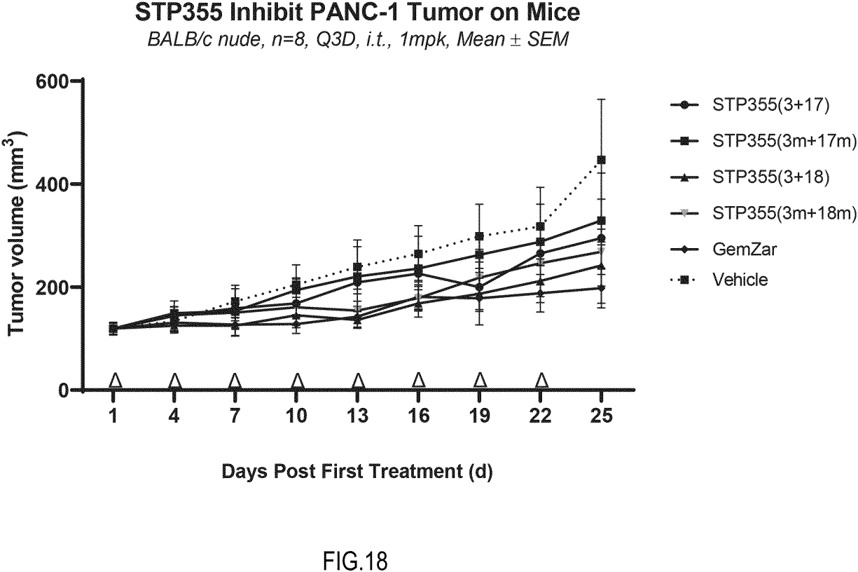

BALB/c nude mice were subcutaneously inoculated with human pancreatic cancer PANC-1 cells on the back, each 4×10⁶/0.2 ml. When the average tumor volume reached 120 mm³, the mice were divided into 6 groups, 8 mice/group. STP355 was administered intratumorally at a dose of 1 mg/k, once every 3 days, for a total of 8 administrations, and gemcitabine (GemZar) (60 mg/kg) was administered as a positive control, intraperitoneally, once every 3 days, a total of 8 doses. Tumor volume and body weight of mice were measured periodically and analyzed by the software GraphPad Prism9. Mean±SE. FIG. 18 shows that each administration group has a certain inhibitory effect on PANC-1 tumor, and the effect is comparable to that of gemcitabine, and there is no difference between the combination drug and the modified drug.

### Example 16. Comparative Stability Test of Modified Drug and Unmodified Drug in C57BL/6J Mice

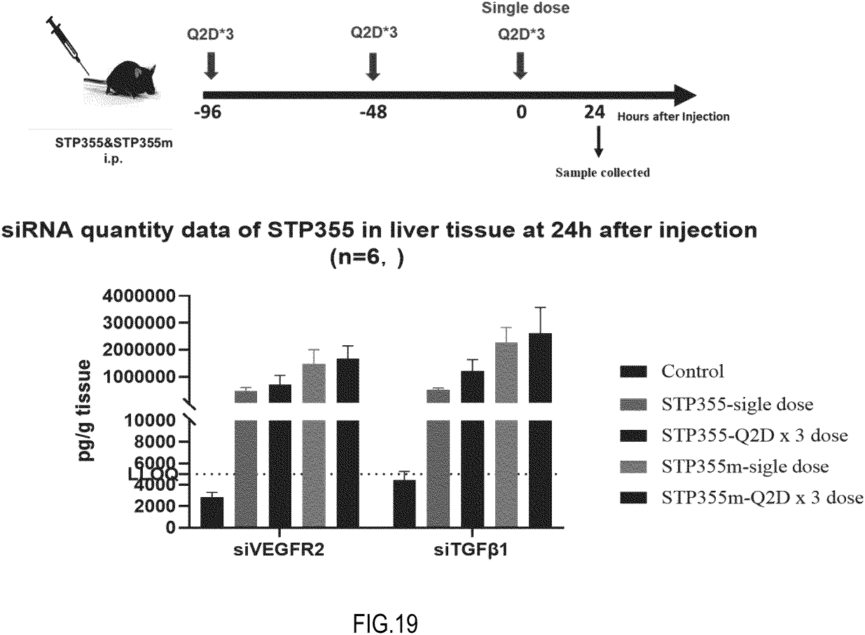

C57BL/6J mice were divided into 5 groups, 6 mice/group. STP355 (3+17) and STP355 (3m+17m) in Example 15 were used to compare the effects of unmodified drug STP355 and modified drug STP355m in mice. For stability, Control is the blank control group, the single dose group (single dose) is administered once, and the repeated administration group (Q2D × 3 doses) is administered once every two days and administered three times. Sampling 24h after the last administration, real-time fluorescence quantitative PCR method was used to determine the content of TGF-β1 siRNA and VEGF-R2 siRNA in liver tissue. FIG. 19 shows that both modified and unmodified drugs could detect higher levels of VEGF-R2 siRNA and TGF-β1 siRNA in liver tissue after i.p. administration. Under the same administration conditions, the residual amount of siRNA in the modified group was higher than that in the unmodified group; in the modified group, the residual amount of siRNA in the repeated administration was higher than that in the single administration; and the residual amount of siRNA in the unmodified group was also higher than that in the single administration. It shows that after modification, siRNA has better stability in vivo.

Human and mouse mRNA molecules encode proteins that are substantially identical in structure or function. Thus, the efficacy and toxicity responses observed in mouse disease models provide a good understanding of what will happen in humans. More importantly, the siRNA molecules tested in mouse models are good candidates for pharmaceutical formulations in humans. In the above examples, the STP355 drug adopts the homologous siRNA of human and mouse.

While this disclosure describes certain examples of such compositions and methods, and numerous details have been set forth for illustrative purposes, it will be apparent to those skilled in the art that these compositions and methods are susceptible to other example’s affect, and certain details may be changed from the examples described herein without departing from the underlying principles of the present disclosure.

### REFERENCES

Adam B. Glick/ Margo M. Lee, Nadine Darwiche, Ashok B. Kulkarni/ Stefan Karlsson,^and Stuart H. Yuspa, (1994) “Targeted deletion of the TGF-pi gene causes rapid progression to squamous cell carcinoma”. GENES & DEVELOPMENT 8:2429-2440.

Annes, J. P., Munger, J. S. & Rifkin, D. B. (2003) Making sense of latent TGF beta activation. J. Cell Sci. 116, 217-224.

Algire, G.H., Chalkley, H.W., Legallais, F.Y., and Park, H.D. (1945) Vasculae reactions of normal and malignant tissues in vivo. I. Vascular reactions of mice to wounds and to normal and neoplastic transplants. J. Natl. Cancer Inst. 6, 73-85.

Akatsu Y, Yoshimatsu Y, Tomizawa T, Takahashi K, Katsura A, Miyazono K, and Watabe T. (2017) Dual targeting of vascular endothelial growth factor and bone morphogenetic protein-9/10 impairs tumor growth through inhibition of angiogenesis. Cancer Sci 108: 151-155

Ide, A.G., Baker, N.H., and Warren, S.L. (1939) Vascularization of the Brown Pearce rabbit epithelioma transplant as seen in the transparent ear chamber. Am. J. Roentgenol. 42, 891-899.

Batlle E, Massague J. (2019) Transforming growth factor-β signaling in immunity and cancer. Immunity 50:924-40.

Bergers G, Hanahan D. (2008) Modes of resistance to anti-angiogenic therapy. Nat Rev Cancer 8: 592-603

Bierie B. and H. L. Moses, (2006) “Tumour microenvironment: TGFB: the molecular Jekyll and Hyde of cancer,” Nature Reviews Cancer, vol. 6, no. 7, pp. 506-520.

Byers L.A. and Heymach J.V. (2007) Dual Targeting of the Vascular Endothelial Growth Factor and Epidermal Growth Factor Receptor Pathways: Rationale and Clinical Applications for Non-Small-Cell Lung Cancer. Clinical Lung Cancer, 8: S79-S85

Crane, J. L. & Cao, X. (2014) Bone marrow mesenchymal stem cells and TGF-beta signaling in bone remodeling. J. Clin. Invest. 124, 466-472.

David JM, Dominguez C, McCampbell KK, et al. A novel bifunctional anti-PD-L1/TGF-β trap fusion protein (M7824) efficiently reverts mesenchymalization of human lung cancer cells. Oncoimmunology 017;6:e1349589.

Diaz-Valdes N., Basagoiti M., Dotor J. et al. (2011) “Induction of monocyte chemoattractant protein-1 and interleukin-10 by TGFβ1 inmelanoma enhances tumor infiltration and immunosuppression,” Cancer Research, vol. 71, no. 3, pp. 812-821.

Dijke P. ten, H. Yamashita, H. Ichijo et al. (1994) “Characterization of type I receptors for transforming growth factor-β and activin,” Science, vol. 264, no. 5155, pp. 101-104.

Folkman, J. (1971). Tumor angiogenesis: therapeutic implications. N. Engl. J. Med. 285, 1182-1186.

Ferrara, N., Hillan, K.J., Gerber, H.P., and Novotny, W. (2004). Discovery and development of bevacizumab, an anti-VEGF antibody for treating cancer. Nat. Rev. Drug Discov. 3, 391-400.

Ferrara N, Binding to the Extracellular Matrix and Proteolytic Processing: Two Key Mechanisms Regulating Vascular Endothelial Growth Factor Action Mol Biol Cell. 2010 Mar 1; 21(5): 687-690.

Ferrara N., Anthony P Adamis, (2016), Ten years of anti-vascular endothelial growth factor therapy. Nat Rev Drug Discov. 15(6):385-403.

Fontenot, JD, Gavin, MA, and Rudensky, AY. (2003) “Foxp3 programs the development and function of CD4+CD25+ regulatory T cells,” Nature Immunology, vol. 4, no. 4, pp. 330-336, 2003.

Gerber Hans-Peter, Napoleone Ferrara, (2005) Pharmacology and pharmacodynamics of bevacizumab as monotherapy or in combination with cytotoxic therapy in preclinical studies Cancer Res, 65(3):671-80.

Gold LI. (1999); The role for transforming growth factor-beta (TGF-beta) in human cancer. Crit Rev Oncog. 10(4):303-360.

Grenga I, Donahue RN, Gargulak ML, et al. (2018) Anti-PD-L1/TGFβR2 (M7824) fusion protein induces immunogenic modulation of human urothelial carcinoma cell lines, rendering them more susceptible to immune-mediated recognition and lysis. Urol Oncol;36:93. el-11. Hagedorn, H. G., Bachmeier, B. E. & Nerlich, A. G. (2001) Synthesis and degradation of basement membranes and extracellular matrix and their regulation by TGF-beta in invasive carcinomas (review). Int. J. Oncol. 18, 669-681.

Hanne Lind, Sofia R Gameiro, Caroline Jochems, Renee N. Donahue, Julius Strauss, James L Gulley, Claudia Palena, Jeffrey Schlom. (2020) Dual targeting of TGF-β and PD-L1 via a bifunctional anti-PD-L1/TGF-βRII agent: status of preclinical and clinical advances. J Immunother Cancer;8:e000433.

Hasegawa, Y. et al. (2001) Transforming growth factor-betal level correlates with angiogenesis, tumor progression, and prognosis in patients with non-small cell lung carcinoma. Cancer 91, 964-971.

Hugo W., Zaretsky J.M., Sun L. et al. (2016) “Genomic and Transcriptomic Features of Response to Anti-PD-1 Therapy in Metastatic Melanoma,” Cell, vol. 165, no. 1, pp. 35-44. Jain, R.K. (2003). Molecular regulation of vessel maturation. Nat. Med. 9, 685-693.

Jayson G.C., Robert Kerbel, Lee M Ellis, Adrian L Harris, (2016) Antiangiogenic therapy in oncology: current status and future directions. Lancet, 388(10043):518-29

Koh YJ, Kim HZ, Hwang SI et al. Double antiangiogenic protein, DAAP, targeting VEGF-A and angiopoietins in tumor angiogenesis, metastasis, and vascular leakage. Cancer Cell 2010; 18: 171-84.

Ikushima H. and K. Miyazono, “TGFbeta signalling: a complex web in cancer progression,” Nature Reviews Cancer, vol. 10, no. 6, pp. 415-424, 2010

de Jong, J. S., van Diest, P. J., van der Valk, P. & Baak, J. P. Expression of growth factors, growth-inhibiting factors, and their receptors in invasive breast cancer. II: Correlations with proliferation and angiogenesis. J. Pathol. 184, 53-57 (1998).

Kang, Y. et al. A multigenic program mediating breast cancer metastasis to bone. Cancer Cell 3, 537-549 (2003).

Keski-Oja, J., Lyons, R.M., and Moses, H.L. (1987). Immunodetection and modulation of cellular growth with antibodies against native transforming growth factor-b1. Cancer Res.47, 6451-6458).

Kerbel, R.S. (2008). Tumor angiogenesis. N. Engl. J. Med. 358, 2039-2049.

Li J.J., Keng-Li Lan, Shun-Fu Chang, Ya-Fen Chen, Wen-Chun Tsai, Pei-Hsun Chiang, Meng-Han Lin, Wolfgang B. Fischer, Yi-Sheng Shih, Sang-Hue Yen, Ren-Shyan Liu, Yeou-Guang Tsay, Hsin-Ell Wang, and Cheng Allen Chang. Development and Characterization of the Recombinant Human VEGF-EGF Dual-Targeting Fusion Protein as a Drug Delivery System. Bioconjugate Chem. 2015, 26, 12, 2481-2496.

Lu SL, Shi-Long Lu, Douglas Reh, Allen G Li, Jennifer Woods, Christopher L Corless, Molly Kulesz-Martin, Xiao-Jing Wang; 2004; Overexpression of transforming growth factor beta1 in head and neck epithelia results in inflammation, angiogenesis, and epithelial hyperproliferation. Cancer Res. 64(13):4405-4410.

Nagy J A, S-H Chang, A M Dvorak, and H F Dvorak,(2009) Why are tumour blood vessels abnormal and why is it important to know? Br J Cancer. 100(6): 865-869

Nakamura, S., Yaguchi, T., Kawamura N. et al., “TGF-β1 in Tumor Microenvironments Induces Immunosuppression in the Tumors and Sentinel Lymph Nodes and Promotes Tumor Progression,” Journal of Immunotherapy, vol. 37, no. 2, pp. 63-72, 2014.

Padua, D. & Massague, J. Roles of TGF beta in metastasis. Cell Res. 19, 89-102 (2009). Papageorgis P., K. Cheng, S. Ozturk et al. (2011); “Smad4 inactivation promotes malignancy and drug resistance of colon cancer,” Cancer Research, vol. 71, no. 3, pp. 998-1008,. Pertovaara, L. et al. Vascular endothelial growth factor is induced in response to transforming growth factor-beta in fibroblastic and epithelial cells. J. Biol. Chem. 269, 6271-6274 (1994). Roberts AB, Wakefield LM. (2003); The two faces of transforming growth factor beta in carcinogenesis. Proc Natl Acad Sci U S A 100(15):8621-8623.

Schadendorf D., C. Gawlik, U. Haney, H. Ostmeier, L. Suter, and B. M. Czarnetzki, “Tumour progression and metastatic behaviour in vivo correlates with integrin expression on melanocytic tumours,” The Journal of Pathology, vol. 170, no. 4, pp. 429-434, 1993.

Seoane, J., “Escaping from the TGFβ anti-proliferative control,” Carcinogenesis, vol. 27, no. 11, pp. 2148-2156, 2006.

Tai K. and C. Wang, “Using Adenovirus Armed Short Hairpin RNA Targeting Transforming Growth Factor β1 Inhibits Melanoma Growth and Metastasis in an Ex Vivo Animal Model,” Annals of Plastic Surgery, p. 1, 2018.

Tuxhorn, J. A., McAlhany, S. J., Yang, F., Dang, T. D. & Rowley, D. R. Inhibition of transforming growth factor-beta activity decreases angiogenesis in a human prostate cancer-reactive stroma xenograft model. Cancer Res. 62, 6021-6025 (2002).

Wrana J. L., L. Attisano, R.Wieser, F. Ventura, and J. Massague, (1994) “Mechanism of activation of the TGF-β receptor,” Nature, vol. 370, no. 6488, pp. 341-347,.

Yamagiwa, S. J. D. Gray, S. Hashimoto, and D. A. Horwitz, “A Role for TGF-beta in the Generation and Expansion of CD4+CD25+ Regulatory T Cells from Human Peripheral Blood,” The Journal of Immunology, vol. 166, no. 12, pp. 7282-7289, 2001.

What is claimed is:

1. A nanoparticle composition comprising a first siRNA duplex that targets VEGFR2, a second RNA duplex that targets TGFβ1 and a pharmaceutically acceptable carrier.

2. The composition according to claim 1, wherein the pharmaceutical carrier is selected from the group consisting of a branched peptide, a polymer, a lipid, and a micelle.

3. The composition according to claim 1, wherein said carrier is a branched HKP polypeptide.

4. A nanoparticle composition according to claim 1, said siRNA that targets VEGFR2 has a sequence selected from the group consisting of SEQ ID Nos. 41-130, and said molecule that targets TGFβ1 has a sequence selected from the group consisting of SEQ ID Nos. 1-40.

5. A nanoparticle composition according to claim 1, said siRNA that targets VEGFR2 has a modified sequence selected from the group consisting of SEQ ID Nos. 131-146, and said molecule that targets TGFβ1 has a modified sequence selected from the group consisting of SEQ ID Nos. 147-154.

6. A composition according to claim 1 wherein said siRNA that targets VEGFR2 has a sequence as set forth in SEQ. ID NO: 60 or SEQ. ID NO: 61, and said siRNA molecule that targets TGFβ1 has a sequence as set forth in SEQ. ID NO: 23 or SEQ. ID NO: 26.

7. The composition according to claim 1 comprising an HKP (+H) polypeptide.

8. A method for treating a disease in a subject comprising administering to said subject a therapeutically effective amount of a composition according to claim 1.

9. A method of attenuating the advancement of a proliferative disease by administering the composition of claim 1 to a subject in need of such treatment.

10. The method of claim 9 wherein the proliferative disease is selected from the group consisting of breast cancers, colon cancers, pancreatic cancers, and diseases characterized by abnormal angiogenesis, wherein said diseases characterized by abnormal angiogenesis are selected from the group consisting of AMD, diabetic angiopathy, and organ fibroses.

11. The method of claim 10, wherein the subject is a mammal.

12. The method of claim 11, wherein the subject is a human.


SEQUENCE LISTING (154 sequences)

SEQ ID NO: 1 - DNA, 21 bp, synthetic construct
  misc_feature 1..21: note = Synthetic oligonucleotide with dTdT at 3' end
  source 1..21: mol_type = other DNA
  misc_feature 20..21: note = DNA
  misc_feature 1..19: note = RNA
gcagagtacacacagcatatt

SEQ ID NO: 2 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tatgctgtgtgtactctgctt

SEQ ID NO: 3 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ccagaaatacagcaacaattt

SEQ ID NO: 4 - DNA, 21 bp, synthetic construct
  misc_feature 1..19: note = Synthetic oligonucleotide; antisense
  source 1..21: mol_type = other DNA
  misc_feature 20..21: note = DNA
  misc_feature 1..19: note = RNA
attgttgctgtatttctggtt

SEQ ID NO: 5 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcaacaattcctggcgatatt

SEQ ID NO: 6 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tatcgccaggaattgttgctt

SEQ ID NO: 7 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cctgtgacagcagggataatt

SEQ ID NO: 8 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttatccctgctgtcacaggtt

SEQ ID NO: 9 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gagtacacacagcatatattt

SEQ ID NO: 10 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atatatgctgtgtgtactctt

SEQ ID NO: 11 - DNA, 21 bp, synthetic construct
  misc_feature 1..21: note = Synthetic oligonucleotide
  source 1..21: mol_type = other DNA
  misc_feature 20..21: note = DNA
  misc_feature 1..19: note = RNA
gactcgccagagtggttattt

SEQ ID NO: 12 - DNA, 21 bp, synthetic construct
  misc_feature 1..21: note = Synthetic oligonucleotide; antisense
  source 1..21: mol_type = other DNA
  misc_feature 20..21: note = DNA
  misc_feature 1..19: note = RNA
ataaccactctggcgagtctt

SEQ ID NO: 13 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcgtgctaatggtggaaactt

SEQ ID NO: 14 - DNA, 21 bp, synthetic construct
  misc_feature 1..21: note = synthetic oligonucleotide; antisense
  source 1..21: mol_type = other DNA
  misc_feature 20..21: note = RNA
  misc_feature 1..19: note = RNA
gtttccaccattagcacgctt

SEQ ID NO: 15 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcagggataacacactgcatt

SEQ ID NO: 16 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tgcagtgtgttatccctgctt

SEQ ID NO: 17 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggacatcaacgggttcacttt

SEQ ID NO: 18 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
agtgaacccgttgatgtcctt

SEQ ID NO: 19 - DNA, 21 bp, synthetic construct
  misc_feature 1..21: note = synthetic oligonucleotide
  source 1..21: mol_type = other DNA
  misc_feature 20..21: note = deoxythymidine dinucleotide (dTdT)
  misc_feature 1..19: note = RNA
ccaccattcatggcatgaatt

SEQ ID NO: 20 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttcatgccatgaatggtggtt

SEQ ID NO: 21 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
tcgacatggagctggtgaatt

SEQ ID NO: 22 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
atcgacatggagctggtgatt

SEQ ID NO: 23 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
aactattgcttcagctccatt

SEQ ID NO: 24 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
accaactattgcttcagcttt

SEQ ID NO: 25 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
tgcggcagctgtacattgatt

SEQ ID NO: 26 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cggcagctgtacattgacttt

SEQ ID NO: 27 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggcagctgtacattgactttt

SEQ ID NO: 28 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
aagggctaccatgccaacttt

SEQ ID NO: 29 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
agggctaccatgccaactttt

SEQ ID NO: 30 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggctaccatgccaacttcttt

SEQ ID NO: 31 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttcaccagctccatgtcgatt

SEQ ID NO: 32 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tcaccagctccatgtcgattt

SEQ ID NO: 33 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tggagctgaagcaatagtttt

SEQ ID NO: 34 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
agctgaagcaatagttggttt

SEQ ID NO: 35 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tcaatgtacagctgccgcatt

SEQ ID NO: 36 - DNA, 21 bp, synthetic construct
  misc_feature 1..20: note = synthetic oligonucleotide; antisense
  source 1..21: mol_type = other DNA
  misc_feature 20..21: note = DNA
  misc_feature 1..19: note = RNA
agtcaatgtacagctgccgtt

SEQ ID NO: 37 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aagtcaatgtacagctgcctt

SEQ ID NO: 38 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tatcgccaggaattgttgctt

SEQ ID NO: 39 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aagttggcatggtagcccttt

SEQ ID NO: 40 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
agaagttggcatggtagcctt

SEQ ID NO: 41 - DNA, 25 bp, synthetic construct
  misc_feature 1..25: note = synthetic oligonucleotide
  source 1..25: mol_type = other DNA
cctcggtcatttatgtctatgttca

SEQ ID NO: 42 - DNA, 25 bp, synthetic construct
  misc_feature 1..25: note = synthetic oligonucleotide
  source 1..25: mol_type = other DNA
cagatctccatttattgcttctgtt

SEQ ID NO: 43 - DNA, 25 bp, synthetic construct
  misc_feature 1..25: note = synthetic oligonucleotide
  source 1..25: mol_type = other DNA
gaccaacatggagtcgtgtacatta

SEQ ID NO: 44 - DNA, 25 bp, synthetic construct
  misc_feature 1..25: note = synthetic oligonucleotide
  source 1..25: mol_type = other DNA
cccttgagtccaatcacacaattaa

SEQ ID NO: 45 - DNA, 25 bp, synthetic construct
  misc_feature 1..25: note = synthetic oligonucleotide
  source 1..25: mol_type = other DNA
ggaggacttccagggaggaaataaa

SEQ ID NO: 46 - DNA, 25 bp, synthetic construct
  misc_feature 1..25: note = synthetic oligonucleotide
  source 1..25: mol_type = other DNA
catggagcttaagaatgcatccttg

SEQ ID NO: 47 - DNA, 25 bp, synthetic construct
  misc_feature 1..25: note = synthetic oligonucleotide
  source 1..25: mol_type = other DNA
cctggagaatcagacgacaagtatt

SEQ ID NO: 48 - DNA, 25 bp, synthetic construct
  misc_feature 1..25: note = synthetic oligonucleotide
  source 1..25: mol_type = other DNA
ccatgttcttctggctacttcttgt

SEQ ID NO: 49 - DNA, 25 bp, synthetic construct
  misc_feature 1..25: note = synthetic oligonucleotide
  source 1..25: mol_type = other DNA
gaactgaagacaggctacttgtcca

SEQ ID NO: 50 - DNA, 25 bp, synthetic construct
  misc_feature 1..25: note = synthetic oligonucleotide
  source 1..25: mol_type = other DNA
ccaagtgattgaagcagatgccttt

SEQ ID NO: 51 - DNA, 25 bp, synthetic construct
  misc_feature 1..25: note = synthetic oligonucleotide
  source 1..25: mol_type = other DNA
tcattcatattggtcaccatctcaa

SEQ ID NO: 52 - DNA, 25 bp, synthetic construct
  misc_feature 1..25: note = synthetic oligonucleotide
  source 1..25: mol_type = other DNA
gagttcttggcatcgcgaaagtgta

SEQ ID NO: 53 - DNA, 25 bp, synthetic construct
  misc_feature 1..25: note = synthetic oligonucleotide
  source 1..25: mol_type = other DNA
cagcaggaatcagtcagtatctgca

SEQ ID NO: 54 - DNA, 25 bp, synthetic construct
  misc_feature 1..25: note = synthetic oligonucleotide
  source 1..25: mol_type = other DNA
cagtggtatggttcttgcctcagaa

SEQ ID NO: 55 - DNA, 25 bp, synthetic construct
  misc_feature 1..25: note = synthetic oligonucleotide
  source 1..25: mol_type = other DNA
aagcagggagtctgtggcatctgaa

SEQ ID NO: 56 - DNA, 25 bp, synthetic construct
  misc_feature 1..25: note = synthetic oligonucleotide
  source 1..25: mol_type = other DNA
ccacactgagctctcctcctgttta

SEQ ID NO: 57 - DNA, 25 bp, synthetic construct
  misc_feature 1..25: note = synthetic oligonucleotide
  source 1..25: mol_type = other DNA
cctacggaccgttaagcgggccaat

SEQ ID NO: 58 - DNA, 25 bp, synthetic construct
  misc_feature 1..25: note = synthetic oligonucleotide
  source 1..25: mol_type = other DNA
gcatctcatctgttacagcttccaa

SEQ ID NO: 59 - DNA, 25 bp, synthetic construct
  misc_feature 1..25: note = synthetic oligonucleotide
  source 1..25: mol_type = other DNA
gcatctcatctgttacagcttccaa

SEQ ID NO: 60 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcctagtgtttctcttgattt

SEQ ID NO: 61 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggtccatttcaaatctcaatt

SEQ ID NO: 62 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cctgatggtaacagaattttt

SEQ ID NO: 63 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gggaatacccttcttcgaatt

SEQ ID NO: 64 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcatcagcataagaaactttt

SEQ ID NO: 65 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ccaagaagaacagcacatttt

SEQ ID NO: 66 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gctgacatgtacggtctattt

SEQ ID NO: 67 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcatcacatccactggtattt

SEQ ID NO: 68 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = DNA
cccatacccttgtgaagaatt

SEQ ID NO: 69 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cctcacatggtacaagctttt

SEQ ID NO: 70 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ccttatgatgccagcaaattt

SEQ ID NO: 71 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggaattgacaagacagcaatt

SEQ ID NO: 72 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gctcctgaagatctgtatatt

SEQ ID NO: 73 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcacgaaatatcctcttattt

SEQ ID NO: 74 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gcctccctttgaaatggattt

SEQ ID NO: 75 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cctcacctgtttcctgtattt

SEQ ID NO: 76 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggactggctttggcccaattt

SEQ ID NO: 77 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggaaaaaacaaaactgtaatt

SEQ ID NO: 78 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggaccgttaagcgggccaatt

SEQ ID NO: 79 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gggaactgaagacaggctatt

SEQ ID NO: 80 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ggcatggagttcttggcattt

SEQ ID NO: 81 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
cctcacctgtttcctgtattt

SEQ ID NO: 82 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
ccaaattccattatgacaatt

SEQ ID NO: 83 - DNA, 21 bp, synthetic construct
  misc_feature 1..21: note = synthetic oligonucleotide
  source 1..21: mol_type = other DNA
  misc_feature 20..21: note = DNA
  misc_feature 1..19: note = RNA
gccccaggaaggaaatgattt

SEQ ID NO: 84 - DNA, 25 bp, synthetic construct
  misc_feature 1..25: note = synthetic oligonucleotide; antisense
  source 1..25: mol_type = other DNA
tgaacatagacataaatgaccgagg

SEQ ID NO: 85 - DNA, 25 bp, synthetic construct
  misc_feature 1..25: note = synthetic oligonucleotide; antisense
  source 1..25: mol_type = other DNA
aacagaagcaataaatggagatctg

SEQ ID NO: 86 - DNA, 25 bp, synthetic construct
  misc_feature 1..25: note = synthetic oligonucleotide; antisense
  source 1..25: mol_type = other DNA
taatgtacacgactccatgttggtc

SEQ ID NO: 87 - DNA, 25 bp, synthetic construct
  misc_feature 1..25: note = synthetic oligonucleotide; antisense
  source 1..25: mol_type = other DNA
ttaattgtgtgattggactcaaggg

SEQ ID NO: 88 - DNA, 25 bp, synthetic construct
  misc_feature 1..25: note = synthetic oligonucleotide; antisense
  source 1..25: mol_type = other DNA
tttatttcctccctggaagtcctcc

SEQ ID NO: 89 - DNA, 25 bp, synthetic construct
  misc_feature 1..25: note = synthetic oligonucleotide; antisense
  source 1..25: mol_type = other DNA
caaggatgcattcttaagctccatg

SEQ ID NO: 90 - DNA, 25 bp, synthetic construct
  misc_feature 1..25: note = synthetic oligonucleotide; antisense
  source 1..25: mol_type = other DNA
aatacttgtcgtctgattctccagg

SEQ ID NO: 91 - DNA, 25 bp, synthetic construct
  misc_feature 1..25: note = synthetic oligonucleotide; antisense
  source 1..25: mol_type = other DNA
acaagaagtagccagaagaacatgg

SEQ ID NO: 92 - DNA, 25 bp, synthetic construct
  misc_feature 1..25: note = synthetic oligonucleotide; antisense
  source 1..25: mol_type = other DNA
tggacaagtagcctgtcttcagttc

SEQ ID NO: 93 - DNA, 25 bp, synthetic construct
  misc_feature 1..25: note = synthetic oligonucleotide; antisense
  source 1..25: mol_type = other DNA
aaaggcatctgcttcaatcacttgg

SEQ ID NO: 94 - DNA, 25 bp, synthetic construct
  misc_feature 1..25: note = synthetic oligonucleotide; antisense
  source 1..25: mol_type = other DNA
ttgagatggtgaccaatatgaatga

SEQ ID NO: 95 - DNA, 25 bp, synthetic construct
  misc_feature 1..25: note = synthetic oligonucleotide; antisense
  source 1..25: mol_type = other DNA
tacactttcgcgatgccaagaactc

SEQ ID NO: 96 - DNA, 25 bp, synthetic construct
  misc_feature 1..25: note = synthetic oligonucleotide; antisense
  source 1..25: mol_type = other DNA
tgcagatactgactgattcctgctg

SEQ ID NO: 97 - DNA, 25 bp, synthetic construct
  misc_feature 1..25: note = synthetic oligonucleotide; antisense
  source 1..25: mol_type = other DNA
ttctgaggcaagaaccataccactg

SEQ ID NO: 98 - DNA, 25 bp, synthetic construct
  misc_feature 1..25: note = synthetic oligonucleotide; antisense
  source 1..25: mol_type = other DNA
ttcagatgccacagactccctgctt

SEQ ID NO: 99 - DNA, 25 bp, synthetic construct
  misc_feature 1..25: note = synthetic oligonucleotide; antisense
  source 1..25: mol_type = other DNA
taaacaggaggagagctcagtgtgg

SEQ ID NO: 100 - DNA, 25 bp, synthetic construct
  misc_feature 1..25: note = synthetic oligonucleotide; antisense
  source 1..25: mol_type = other DNA
attggcccgcttaacggtccgtagg

SEQ ID NO: 101 - DNA, 25 bp, synthetic construct
  misc_feature 1..25: note = synthetic oligonucleotide; antisense
  source 1..25: mol_type = other DNA
ttggaagctgtaacagatgagatgc

SEQ ID NO: 102 - DNA, 25 bp, synthetic construct
  misc_feature 1..25: note = synthetic oligonucleotide; antisense
  source 1..25: mol_type = other DNA
atgccaagaactccatgcccttagc

SEQ ID NO: 103 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atcaagagaaacactaggctt

SEQ ID NO: 104 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttgagatttgaaatggacctt

SEQ ID NO: 105 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aaattctgttaccatcaggtt

SEQ ID NO: 106 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttcgaagaagggtattccctt

SEQ ID NO: 107 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aagtttcttatgctgatgctt

SEQ ID NO: 108 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aatgtgctgttcttcttggtt

SEQ ID NO: 109 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atagaccgtacatgtcagctt

SEQ ID NO: 110 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ataccagtggatgtgatgctt

SEQ ID NO: 111 - DNA, 21 bp, synthetic construct
  misc_feature 1..21: note = synthetic oligonucleotide; antisense
  source 1..21: mol_type = other DNA
  misc_feature 1..19: note = RNA
  misc_feature 20..21: note = DNA
ttcttcacaagggtatgggtt

SEQ ID NO: 112 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
aagcttgtaccatgtgaggtt

SEQ ID NO: 113 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atttgctggcatcataaggtt

SEQ ID NO: 114 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttgctgtcttgtcaattcctt

SEQ ID NO: 115 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tatacagatcttcaggagctt

SEQ ID NO: 116 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ataagaggatatttcgtgctt

SEQ ID NO: 117 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atccatttcaaagggaggctt

SEQ ID NO: 118 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atacaggaaacaggtgaggtt

SEQ ID NO: 119 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
attgggccaaagccagtcctt

SEQ ID NO: 120 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttacagttttgttttttcctt

SEQ ID NO: 121 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttggcccgcttaacggtcctt

SEQ ID NO: 122 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
tagcctgtcttcagttccctt

SEQ ID NO: 123 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atgccaagaactccatgcctt

SEQ ID NO: 124 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atacaggaaacaggtgaggtt

SEQ ID NO: 125 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
ttgtcataatggaatttggtt

SEQ ID NO: 126 - DNA, 21 bp, synthetic construct
  misc_feature 1..21: note = synthetic oligonucleotide; antisense
  source 1..21: mol_type = other DNA
  misc_feature 20..21: note = DNA
  misc_feature 1..19: note = RNA
atcatttccttcctggggctt

SEQ ID NO: 127 - DNA, 21 bp, synthetic construct
  misc_feature 1..21: note = synthetic oligonucleotide; antisense
  source 1..21: mol_type = other DNA
aagctcagcacacagaaagac

SEQ ID NO: 128 - DNA, 21 bp, synthetic construct
  misc_feature 1..21: note = synthetic oligonucleotide; antisense
  source 1..21: mol_type = other DNA
gtctttctgtgtgctgagctt

SEQ ID NO: 129 - DNA, 21 bp, synthetic construct
  misc_feature 1..21: note = synthetic oligonucleotide
  source 1..21: mol_type = other DNA
aatgcggcggtggtgacagta

SEQ ID NO: 130 - DNA, 21 bp, synthetic construct
  misc_feature 1..21: note = synthetic oligonucleotide; antisense
  source 1..21: mol_type = other DNA
tactgtcaccaccgccgcatt

SEQ ID NO: 131 - DNA, 21 bp, synthetic construct
  misc_feature 1..21: note = modified synthetic oligonucleotide as indicated in features, withdTdT on 3' end
  modified_base 1..6: mod_base = OTHER; note = 2-O-methyl
  modified_base 7..9: mod_base = OTHER; note = 2-Fluoro
  modified_base 10..19: mod_base = OTHER; note = 2-O-methyl
  source 1..21: mol_type = other DNA
  misc_feature 20..21: note = DNA
  misc_feature 1..19: note = RNA
gcctagtgtttctcttgattt

SEQ ID NO: 132 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_RNA 1..19: note = RNA
  misc_feature 20..21: note = DNA
ggtccatttcaaatctcaatt

SEQ ID NO: 133 - DNA, 25 bp, synthetic construct
  misc_feature 1..25: note = modified synthetic olligonucleotide
  modified_base 1..12: mod_base = OTHER; note = 2-O-methyl
  modified_base 13..15: mod_base = OTHER; note = 2 fluoro
  modified_base 16..25: mod_base = OTHER; note = 2-O-methyl
  source 1..25: mol_type = other DNA
gaccaacatggagtcgtgtacatta

SEQ ID NO: 134 - DNA, 25 bp, synthetic construct
  misc_feature 1..25: note = modified synthetic oligonucleotide
  modified_base 1..12: mod_base = OTHER; note = 2-O-methyl
  modified_base 13..15: mod_base = OTHER; note = 2-fluoro
  modified_base 16..25: mod_base = OTHER; note = 2-O-methyl
  source 1..25: mol_type = other DNA
cccttgagtccaatcacacaattaa

SEQ ID NO: 135 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 1..19: note = RNA
  misc_feature 20..21: note = DNA
ggactggctttggcccaattt

SEQ ID NO: 136 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 1..19: note = RNA
  misc_feature 20..21: note = DNA
ggaaaaaacaaaactgtaatt

SEQ ID NO: 137 - DNA, 21 bp, synthetic construct
  misc_feature 1..21: note = modified synthetic oligonucleotide
  modified_base 1..6: mod_base = OTHER; note = 2-O-methyl
  modified_base 7: mod_base = OTHER; note = 2-fluoro
  modified_base 10..19: mod_base = OTHER; note = 2-O-methyl
  source 1..21: mol_type = other DNA
  misc_feature 1..19: note = RNA
  misc_feature 20..21: note = DNA
gcatcagcataagaaactttt

SEQ ID NO: 138 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 1..19: note = RNA
  misc_feature 20..21: note = DNA
gctgacatgtacggtctattt

SEQ ID NO: 139 - DNA, 21 bp, synthetic construct
  misc_feature 1..21: note = modified synthetic oligonucleotide
  modified_base 1: mod_base = OTHER; note = phosphorylation, 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2-fluorocytidine
  modified_base 15..19: mod_base = OTHER; note = 2-O-methyl
  source 1..21: mol_type = other DNA
  misc_feature 1..19: note = RNA
  misc_feature 20..21: note = DNA
atcaagagaaacactaggctt

SEQ ID NO: 140 - DNA, 21 bp, synthetic construct
  misc_feature 1..21: note = modified synthetic oligonucleotide
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluorouridine
  modified_base 15..19: mod_base = OTHER; note = 2-O-methyl
  source 1..21: mol_type = other DNA
  misc_feature 1..19: note = RNA
  misc_feature 20..21: note = DNA
ttgagatttgaaatggacctt

SEQ ID NO: 141 - DNA, 25 bp, synthetic construct
  misc_feature 1..25: note = modified synthetic oligonucleotide as indicated; antisense
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..25: mod_base = OTHER; note = 2-O-methyl
  source 1..25: mol_type = other DNA
taatgtacacgactccatgttggtc

SEQ ID NO: 142 - DNA, 25 bp, synthetic construct
  misc_feature 1..25: note = modified synthetic oligonucleotide as indicated; antisense
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..25: mod_base = OTHER; note = 2-O-methyl
  source 1..25: mol_type = other DNA
ttaattgtgtgattggactcaaggg

SEQ ID NO: 143 - DNA, 21 bp, synthetic construct
  misc_feature 1..21: note = modified synthetic oligonucleotide
  modified_base 1: mod_base = OTHER; note = phosphorylation, 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
attgggccaaagccagtcctt

SEQ ID NO: 144 - DNA, 21 bp, synthetic construct
  misc_feature 1..21: note = modified synthetic oligonucleotide
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
ttacagttttgttttttcctt

SEQ ID NO: 145 - DNA, 21 bp, synthetic construct
  misc_feature 1..21: note = modified synthetic oligonucleotide
  modified_base 1: mod_base = OTHER; note = phosphorylation, 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
aagtttcttatgctgatgctt

SEQ ID NO: 146 - DNA, 21 bp, synthetic construct
  misc_feature 1..21: note = modified synthetic oligonucleotide
  modified_base 1: mod_base = OTHER; note = phosphorylation, 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
atagaccgtacatgtcagctt

SEQ ID NO: 147 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 20..21: note = DNA
  misc_feature 1..19: note = RNA
aactattgcttcagctccatt

SEQ ID NO: 148 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 20..21: note = DNA
  misc_feature 1..19: note = RNA
cggcagctgtacattgacttt

SEQ ID NO: 149 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 20..21: note = DNA
  misc_feature 1..19: note = RNA
gcagagtacacacagcatatt

SEQ ID NO: 150 - DNA, 21 bp, synthetic construct
  misc_feature 1..21: note = modified synthetic oligonucleotide
  modified_base 1..6: mod_base = OTHER; note = 2-O-methyl
  modified_base 7..9: mod_base = OTHER; note = 2-fluoro
  modified_base 10..19: mod_base = OTHER; note = 2-O-methyl
  source 1..21: mol_type = other DNA
  misc_feature 20..21: note = DNA
  misc_feature 1..19: note = RNA
ggcagctgtacattgactttt

SEQ ID NO: 151 - DNA, 21 bp, synthetic construct
  misc_feature 1..21: note = modified synthetic oligonucleotide
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
tggagctgaagcaatagtttt

SEQ ID NO: 152 - DNA, 21 bp, synthetic construct
  misc_feature 1..21: note = modified synthetic oligonucleotide
  source 1..21: mol_type = other DNA
  misc_feature 20..21: note = DNA
  modified_base 1: mod_base = OTHER; note = phosphorylation; 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2 -fluoro
  modified_base 15..19: mod_base = OTHER; note = 2-O-methyl
  misc_feature 1..19: note = RNA
agtcaatgtacagctgccgtt

SEQ ID NO: 153 - DNA, 21 bp, synthetic construct
  misc_feature 1..21: note = modified synthetic oligonucleotide
  modified_base 1: mod_base = OTHER; note = phosphorylation, um
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
tatgctgtgtgtactctgctt

SEQ ID NO: 154 - DNA, 21 bp, synthetic construct
  misc_feature 1..21: note = modified synthetic oligonucleotide
  modified_base 1: mod_base = OTHER; note = phosphorylation; 2-O-methyl
  modified_base 2: mod_base = OTHER; note = 2-fluoro
  modified_base 3..13: mod_base = OTHER; note = 2-O-methyl
  modified_base 14: mod_base = OTHER; note = 2-fluoro
  modified_base 15..19: mod_base = OTHER; note = 2-O-methyl
  source 1..21: mol_type = other DNA
  misc_feature 20..21: note = DNA
  misc_feature 1..19: note = RNA
aagtcaatgtacagctgcctt